SEQUENCE LISTING (21 sequences)

SEQ ID NO: 1 - protein, 9 aa, Homo sapiens
GYSITSDYA

SEQ ID NO: 2 - protein, 9 aa, Homo sapiens
GYISYSGST

SEQ ID NO: 3 - protein, 9 aa, Homo sapiens
ARGGTYFDY

SEQ ID NO: 4 - protein, 9 aa, Homo sapiens
RASAHINNW

SEQ ID NO: 5 - protein, 7 aa, Homo sapiens
DATSLES

SEQ ID NO: 6 - protein, 9 aa, Homo sapiens
QHYWSTPWT

SEQ ID NO: 7 - protein, 116 aa, Homo sapiens
EVQLQESGPGLVKPSQTLSLTCTVSGYSITSDYAWNWIRQPPGKGLEWIGYISYSGSTNYNPSLKSRVTISRDTSKNQFSLKLSSVTAADTAVYYCARGGTYFDYWGQGTLVTVSS

SEQ ID NO: 8 - protein, 107 aa, Homo sapiens
DVVMTQSPSTLSASVGDRVTITCRASAHINNWLAWYQQKPGKAPKLLISDATSLESGVPSRFSGSGSGKEYTLTISSLQPDDFATYYCQHYWSTPWTFGQGTKVEIK

SEQ ID NO: 9 - protein, 445 aa, Homo sapiens
EVQLQESGPGLVKPSQTLSLTCTVSGYSITSDYAWNWIRQPPGKGLEWIGYISYSGSTNYNPSLKSRVTISRDTSKNQFSLKLSSVTAADTAVYYCARGGTYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 214 aa, Homo sapiens
DVVMTQSPSTLSASVGDRVTITCRASAHINNWLAWYQQKPGKAPKLLISDATSLESGVPSRFSGSGSGKEYTLTISSLQPDDFATYYCQHYWSTPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 274 aa, Homo sapiens
EPHSLRYNLTVLSWDGSVQSGFLAEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETEEWTMPQSSRAQTLAMNIRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPS

SEQ ID NO: 12 - protein, 274 aa, Homo sapiens
EPHSLRYNLMVLSQDGSVQSGFLAEGHLDGQPFLRYDRQKRRAKPQGQWAEDVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPS

SEQ ID NO: 13 - protein, 97 aa, Homo sapiens
QVQLQESGPGLVKPSETLSLTCTVSGGSISSHYWSWIRQPPGKGLEWIGYIYYSGSTNYNPSLKSRVTISVDTSKNQFSLKLSSVTAADTAVYYCAR

SEQ ID NO: 14 - protein, 15 aa, Homo sapiens
YFDYWGQGTLVTVSS

SEQ ID NO: 15 - protein, 98 aa, Homo sapiens
QVQLQESGPGLVKPSQTLSLTCTVSGGSISSGDYYWSWIRQPPGKGLEWIGYIYYSGSTYYNPSLKSRVTIVDTSKNQFSLKLSSVTAADTAVYYCAR

SEQ ID NO: 16 - protein, 95 aa, Homo sapiens
DIQMTQSPSSLSASVGDRVTITCRASQGISNSLAWYQQKPGKAPKLLLYAASRLESGVPSRFSGSGSGTDYTLTISSLQPEDFATYYCQQYYSTP

SEQ ID NO: 17 - protein, 12 aa, Homo sapiens
WTFGQGTKVEIK

SEQ ID NO: 18 - protein, 95 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQYDNLP

SEQ ID NO: 19 - protein, 95 aa, Homo sapiens
DIQMTQSPSTLSASVGDRVTITCRASQSISSWLAWYQQKPGKAPKLLIYDASSLESGVPSRFSGSGSGTEFTLTISSLQPDDFATYYCQQYNSYS

SEQ ID NO: 20 - DNA, 348 bp, Homo sapiens
  source 1..348: mol_type = other DNA
gaagtgcagctgcaggaatctggccctggcctcgtgaagccttcccagaccctgtctctgacctgcaccgtgtccggctactccatcacctccgactacgcctggaactggatccggcagcctcctggcaagggactggaatggatcggctacatctcctactccggctccaccaactacaaccccagcctgaagtccagagtgaccatctcccgggacacctccaagaaccagttctccctgaagctgtcctccgtgaccgccgctgataccgccgtgtactactgtgctagaggcggcacctacttcgactactggggccagggcaccctcgtgaccgtgtcatct

SEQ ID NO: 21 - DNA, 321 bp, Homo sapiens
  source 1..321: mol_type = other DNA
gacgtcgtgatgacccagtccccctccacactgtctgcctctgtgggcgacagagtgaccatcacctgtcgggcctccgcccacatcaacaactggctggcctggtatcagcagaagcccggcaaggcccctaagctgctgatctctgatgccacctccctggaatccggcgtgccctccagattctccggctctggctctggcaaggagtataccctgaccatcagctccctgcagcccgatgacttcgccacctactactgccagcactactggtccaccccctggacctttggccaaggcaccaaggtggaaatcaag

(19) United States
(12) Patent Application Publication
(10) Pub. No.: US 2025/0230246 A1
(43) Pub. Date: Jul. 17, 2025

(54) ANTI-MIC ANTIBODIES WITH VARIANT FC DOMAINS

(71) Applicant: SAMYANG BIOPHARM USA INC., Cambridge, MA (US)

(72) Inventor: Sean Daniel MCKENNA, Cambridge, MA (US)

(73) Assignee: SAMYANG BIOPHARM USA INC., Cambridge, MA (US)

(21) Appl. No.: 18/702,171

(22) PCT Filed: Oct. 25, 2022
(86) PCT No.: PCT/US2022/078634

§ 371 Date: Apr. 17, 2024

Related U.S. Application Data
(60) Provisional application No. 63/272,261, filed on Oct. 27, 2021.

(51) Int. Cl. C07K 16/28; A61K 39/00; A61P 35/00

(52) U.S. Cl. CPC C07K 16/2833; A61P 35/00; A61K 2039/505; C07K 2317/52; C07K 2317/732; C07K 2317/92

(57) ABSTRACT

Provided herein are anti-MIC antibodies comprising one or more Fc domain mutations to eliminate Fc effector functions. Also provided herein are methods of use in treating cancer by administering the anti-MIC antibodies described herein.

### CROSS-REFERENCE TO RELATED APPLICATIONS

This application claims priority to U.S. Provisional Patent Application No. 63/272,261, filed Oct. 27, 2021, which is incorporated by reference herein in its entirety for all purposes.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: SMYG-003_01WO_SeqList_ST26.xml, date recorded: Oct. 13, 2022, file size: 21,568 bytes).

### TECHNICAL FIELD

The present disclosure relates to anti-MIC antibodies with variant Fc domains and uses of such antibodies for treating cancer.

### BACKGROUND

Major Histocompatibility Complex class I chain-related molecules A and B (MICA and MICB, respectively, generally termed MIC) are a family of proteins that bind to NKG2D. NKG2D is an activating immune receptor expressed by natural killer (NK) cells, NKT cells, subsets of gamma-delta T cells, and human CD8 T cells. Binding of MIC to NKG2D on NK cells or T cells leads to, among other effects, NK cell activation and co-stimulation of CD8 and gamma-delta T cells.

MIC family molecules are expressed on tumor cells and are involved in suppression of immune responses to the tumor cells. The MIC protein is found in a membrane bound form, a soluble form (sMIC) that is shed from tumor cells, and an exosomal form, wherein MIC is released from cells in extracellular vesicles. The soluble and exosomal forms are immunosuppressive and can contribute to the pathogenesis of many types of cancer, including carcinomas, such as a solid tumor, e.g., melanoma, prostate cancer, ovarian cancer, cervical cancer, breast cancer, lung cancer, colon cancer, kidney cancer, and head and neck cancer, and hematopoietic cancers, such as lymphomas, multiple myelomas and melanomas. Thus, there remains a need for agents optimized for treatment of MIC+ cancers in humans.

### BRIEF DESCRIPTION OF THE FIGURES

FIG. 1A-FIG. 1D illustrate that SYB-010 and SYB-011 blocked the binding between MICB and NKG2D.

FIG. 2 illustrates the effects of SYB-010, SYB-011, and 3F9 antibodies on NKG2D expression in EV-treated NK cells.

FIG. 3A-FIG. 3C illustrate that SYB-011 was better able to prevent MIC+ EV-induced NKG2D downregulation than SYB-010.

FIG. 4 illustrates SYB-011 prevented MIC detection on NK cells following incubation with MIC+ EVs.

FIG. 5 illustrates the effects of SYB-010, SYB-011, and 3F9 antibodies on NK cell degranulation.

FIG. 6A-FIG. 6D illustrate the effects of SYB-010, SYB-011, and 3F9 antibodies on NK cell lysis of target cells.

FIG. 7A-FIG. 7B illustrate that SYB-010, not SYB-011, was associated with increased NK cell death.

FIG. 8 provides a schematic illustrating the “preventative” and “reversal” assays described in the examples.

FIG. 9 illustrates the effects of SYB-010, SYB-011, and 3F9 antibodies on the reversal of inhibition of NK cell degranulation induced by MIC+ EVs.

FIG. 10 illustrates that SYB-010, not SYB-011, inhibited NK cell lysis of target cells.

FIG. 11 illustrates the effects of SYB-010, SYB-011, and 3F9 antibodies on NKG2D expression on NK cells incubated with tumor cells expressing endogenous MIC rather than MIC+ EVs and tumor cells.

FIG. 12 illustrates the effects of SYB-010 and SYB-011 antibodies on NKG2D expression in EV-treated T cells.

FIG. 13 illustrates that SYB-011 prevented MIC detection on T cells following incubation with MIC+ EVs.

FIG. 14A-FIG. 14B illustrate the effects of SYB-010 and SYB-011 antibodies in vivo.

### SUMMARY

In some embodiments, the present disclosure provides an anti-MIC antibody comprising a heavy chain variable (VH) region, a light chain variable (VL) region, and an Fc domain, wherein the VH region comprises a complementarity determining region HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3, wherein the VL region comprises a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6, wherein the VH region and VL region each comprise a humanized framework region sequence, and wherein the Fc domain comprises the point mutations L234A and L235A according to the EU index. In some embodiments, the Fc domain further comprises a P329G mutation according to the EU index. In some embodiments, the Fc domain comprises the point mutations P329G, L234A, and L235A.

In some embodiments, the VH region comprises the amino acid sequence of SEQ ID NO: 7 and the VL region comprises the amino acid sequence of SEQ ID NO: 8.

In some embodiments, the humanized VH framework region is derived from a human germline gene having the amino acid sequence set forth in IMGT IGHV4-59*11, IGHV4-30-4*01 or IGHV4-30-4*08. In some embodiments, the humanized VL framework region is derived from a human germline gene having the amino acid sequence set forth in IMGT IGKV1-5*01, IGKV1-5*02 or IGKV1-5*03.

In some embodiments, the present disclosure provides an anti-MIC antibody comprising a heavy chain and a light chain, wherein the heavy chain comprises the amino acid sequence of SEQ ID NO: 9 comprising the point mutations L234A and L235A according to the EU index, and the light chain comprises the amino acid sequence of SEQ ID NO: 10.

In some embodiments, the present disclosure provides an anti-MIC antibody comprising a heavy chain and a light chain, wherein the heavy chain comprises the amino acid sequence of SEQ ID NO: 9 comprising the point mutations P329G, L234A, and L235A according to the EU index, and the light chain comprises the amino acid sequence of SEQ ID NO: 10.

In some embodiments, the antibody increases Natural Killer (NK) cell activation compared to an anti-MIC antibody that does not comprise an Fc domain comprising the point mutations P329G, L234A, and/or L235A according to the EU index. In some embodiments, the antibody increases tumor cell lysis compared to an anti-MIC antibody that does not comprise an Fc domain comprising the point mutations P329G, L234A, and/or L235A according to the EU index. In some embodiments, the antibody abrogates MIC+ extracellular vesicles (MIC+ EV)-mediated immune suppression to a greater extent than an anti-MIC antibody that does not comprise an Fc domain comprising the point mutations P329G, L234A, and/or L235A according to the EU index.

In some embodiments, the present disclosure provides a pharmaceutical composition comprising the anti-MIC antibodies described herein.

In some embodiments, the present disclosure provides a nucleic acid encoding an antibody heavy chain comprising the amino acid sequence of SEQ ID NO: 9, wherein the encoded antibody chain further comprises the point mutations (i) L234A and L235A; or (ii) P329G, L234A, and L235A. In some embodiments, the vector further comprises

In some embodiments, the present disclosure provides a vector comprising a nucleic acid encoding an antibody heavy chain. In some embodiments, the vector further comprises a nucleic acid sequence encoding an antibody light chain comprising the amino acid sequence of SEQ ID NO: 10.

In some embodiments, the present disclosure provides a host cell comprising a vector of the present disclosure.

In some embodiments, the present disclosure provides a method of treating a MIC+ cancer, comprising administering to a subject in need thereof a therapeutically effective amount of the anti-MIC antibodies or compositions thereof described herein.

In some embodiments, the present disclosure provides a method of abrogating MIC-positive extracellular vesicle (MIC+ EV) induced immunosuppression in a subject suffering from a cancer, comprising administering a therapeutically effective amount of the anti-MIC antibodies or compositions thereof described herein. In some embodiments, the MIC+ EV induced immunosuppression is decreased expression of NKG2D on NK cells and/or decreased NK cell degranulation.

In some embodiments, the cancer is a carcinoma or a hematologic malignancy. In some embodiments, the carcinoma is a solid tumor selected from melanoma, prostate cancer, ovarian cancer, cervical cancer, breast cancer, lung cancer, colon cancer, kidney cancer, and head and neck cancer. In some embodiments, the hematologic malignancy is a lymphoma or multiple myeloma.

In some embodiments, the method further comprises administering an immunotherapy to the subject. In some embodiments, the immunotherapy comprises an adoptive cell therapy or a checkpoint inhibitor. In some embodiments, the adoptive cell therapy is selected from autologous NK cells, allogeneic NK cells, autologous T cells, CAR-modified T cells, and CAR-modified NK cells. In some embodiments, the checkpoint inhibitor is an antibody that specifically binds to human PD-1, human PD-L1, or human CTLA4. In some embodiments, the checkpoint inhibitor is pembrolizumab, nivolumab, cemiplimab, or ipilimumab. In some embodiments, chemotherapy is not administered to the subject for at least four weeks prior to the administration of the anti-MIC antibody.

In some embodiments, the anti-MIC antibody is administered intravenously. In some embodiments, the anti-MIC antibody is administered in a dose of about 0.1 mg/kg to about 10 mg/kg.

### DETAILED DESCRIPTION

### Overview

Natural killer (NK) cells are an important immune cell population contributing to anti-viral and anti-tumor immune responses. Their activity is tightly regulated by a battery of stimulatory and inhibitory receptors. Natural killer group 2-member D (NKG2D) is one of the well characterized activating receptors. NKG2D is a type II transmembrane, homo-dimeric receptor expressed on the surface of almost all human NK cells, activated CD8 αβ+ T cells, γδ T cells, and NKT cells. Ligand engagement of the NKG2D receptor triggers a potent intracellular signaling cascade via the adaptor DAP10, leading to cytokine secretion and cytolysis of target cells.

A host of NKG2D receptor ligands have been identified, including the MHC class I chain related molecules A and B (MICA/B). MICA and MICB are cell surface glycoproteins encoded by two highly polymorphic genes, that reside in the human HLA class I locus. NKG2D recognition of MICA/B expressed on tumor cells results in NK cell activation and tumor cytolysis.

To escape NKG2D-mediated immune surveillance, tumors can shed MIC from the cell surface via proteolytic cleavage. Shed MICA/B has been hypothesized to dampen the host immune response by inducing the down-regulation of cell surface NKG2D, thereby inhibiting NK cell function, and destabilizing CD3ζ in the TCR/CD3 complex on CD8 T cells.

An alternative MIC-related pathway by which tumor cells promote immune evasion is the release of MIC into exosomes. The MICA5.1 allele family lacks the proteolytic cleavage domain required for MIC shedding from tumor cells. Instead, MICA*008, a member of the MICA5.1 allele family, is released from cells largely on exosomes. Exosomal MICA*008 molecules are also highly immunosuppressive and are able to trigger downregulation of NKG2D from the NK cell surface. In addition, exposure of NK cells to MICA*008-containing exosomes triggers a marked loss of cytotoxic function (Ashiru, Cancer Res Jan. 15, 2010 (70) (2) 481-489; DOI: 10.1158/0008-5472).

MICA*008 is the most frequently expressed MICA allele, with an allele frequency of approximately 42% of Western Europeans (Klussmeier, 2020) and 55% of US Caucasians (Allele Frequency Net Database). It is unclear from published studies whether existing antibodies that bind surface and/or soluble MIC can also bind to and prevent the immunosuppressive actions of exosomal MIC. Thus, there is a need for cancer therapeutics that prevent the potent immune suppression observed with exosomal MIC. The present disclosure meets this need by providing an anti-MIC antibody comprising an Fc domain with 3 point mutations that eliminate Fc receptor binding and abrogate Fc cross-linking—P329G, L234A, and L235A (an “effectorless” anti-MIC antibody). This effectorless anti-MIC antibody, referred to herein as SYB-011, abrogates exosomal MIC-induced NK cell inhibition, therefore enabling treatment of cancer patients expressing membrane-restricted forms of MIC.

### Definitions

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

The terms “decrease,” “reduce,” “reduced”, “reduction”, “decrease,” and “inhibit” are all used herein generally to mean a decrease by a statistically significant amount relative to a reference.

The terms “increased”, “increase” or “enhance” or “activate” are all used herein to generally mean an increase by a statically significant amount relative to a reference.

The term “antibody” refers to an immunoglobulin (Ig) molecule capable of binding to a specific target, such as a carbohydrate, polynucleotide, lipid, or polypeptide, through at least one epitope recognition site located in the variable region of the Ig molecule. As used herein, the term encompasses intact polyclonal or monoclonal antibodies and antigen-binding fragments thereof. For example, a native immunoglobulin molecule is comprised of two heavy chain polypeptides and two light chain polypeptides. Each of the heavy chain polypeptides associate with a light chain polypeptide by virtue of interchain disulfide bonds between the heavy and light chain polypeptides to form two heterodimeric proteins or polypeptides (i.e., a protein comprised of two heterologous polypeptide chains). The two heterodimeric proteins then associate by virtue of additional interchain disulfide bonds between the heavy chain polypeptides to form an immunoglobulin protein or polypeptide.

“Fc region” or “Fc domain” refers to a polypeptide sequence corresponding to or derived from the portion of an antibody that is capable of binding to Fc receptors on cells and/or the C1q component of complement, thereby mediating the effector function of an antibody. Fc stands for “fragment crystalline,” the fragment of an antibody that will readily form a protein crystal. Distinct protein fragments, which were originally described by proteolytic digestion, can define the overall general structure of an immunoglobulin protein. As originally defined in the literature, the Fc region is a homodimeric protein comprising two polypeptides that are associated by disulfide bonds, and each comprising a hinge region, a CH2 domain, and a CH3 domain. However, more recently the term has been applied to the single chain monomer component consisting of CH3, CH2, and at least a portion of the hinge sufficient to form a disulfide-linked dimer with a second such chain. As such, and depending on the context, use of the terms “Fc region” or “Fc domain” will refer herein to either the dimeric form or the individual monomers that associate to form the dimeric protein. For a review of immunoglobulin structure and function, see Putnam, The Plasma Proteins, Vol. V (Academic Press, Inc., 1987), pp. 49-140; and Padlan, Mol. Immunol. 31:169-217, 1994. As used herein, the term Fc domain includes variants of naturally occurring sequences.

The term “immunoglobulin constant region” or “constant region” refers to a peptide or polypeptide sequence that corresponds to or is derived from part or all of one or more constant domains of an immunoglobulin (e.g., CH1, CH2, CH3). In certain embodiments, the constant region does not comprise a CH1 domain. In certain embodiments, the constant domains making up the constant region are human

The terms “light chain variable region” (also referred to as “light chain variable domain” or “VL”) and “heavy chain variable region” (also referred to as “heavy chain variable domain” or “VH”) refer to the variable binding region from an antibody light and heavy chain, respectively. The variable binding regions are made up of discrete, well-defined sub-regions known as “complementarity determining regions” (CDRs) and “framework regions” (FRs).

The term “immunoglobulin light chain constant region” (also referred to as “light chain constant region” or “CL”) is a constant region from an antibody light chain.

The term “immunoglobulin heavy chain constant region” (also referred to as “heavy chain constant region” or “CH”) refers to the constant region from the antibody heavy chain. The CH is further divisible, depending on the antibody isotype into CH1, CH2, and CH3 (IgA, IgD, IgG), or CH1, CH2, CH3, and CH4 domains (IgE, IgM).

As used herein, the term “complementarity determining region” or “CDR” refer to an immunoglobulin (antibody) molecule. There are three CDRs per variable domain: CDR1, CDR2 and CDR3 in the variable domain of the light chain and CDR1, CDR2 and CDR3 in the variable domain of the heavy chain.

In some embodiments, a “hinge” or a “hinge region” refers to a polypeptide derived from an immunoglobulin hinge region and located between an antigen-binding domain (e.g., a ceramide-binding domain) and an immunoglobulin constant region in a polypeptide described herein. A “wild-type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of an antibody. In certain embodiments, a wild type immunoglobulin hinge region sequence is human, and can comprise a human IgG hinge region (e.g., and IgG1, IgG2, IgG3, or IgG4 hinge region).

The terms “isolated” or “partially purified” as used herein refer in the case of a nucleic acid, polypeptide or protein, to a nucleic acid, polypeptide or protein separated from at least one other component (e.g., nucleic acid or polypeptide or protein) that is present with the nucleic acid, polypeptide or protein as found in its natural source and/or that would be present with the nucleic acid, polypeptide or protein when expressed by a cell, or secreted in the case of secreted polypeptides and proteins. A chemically synthesized nucleic acid, polypeptide or protein, or one synthesized using in vitro transcription/translation, is considered “isolated.” The terms “purified” or “substantially purified” refer to an isolated nucleic acid, polypeptide or protein that is at least 95% by weight the subject nucleic acid, polypeptide or protein, including, for example, at least 96%, at least 97%, at least 98%, at least 99% or more.

As used herein, the term “pharmaceutically acceptable” refers to molecular entities and compositions that do not generally produce allergic or other serious adverse reactions when administered using routes well known in the art. Molecular entities and compositions approved by a regulatory agency of the Federal or a state government or listed in the U.S. Pharmacopeia or other generally recognized pharmacopeia for use in animals, and more particularly in humans are considered to be “pharmaceutically acceptable.”

The term “polynucleotide” as referred to herein means single-stranded or double-stranded nucleic acid polymers. In certain embodiments, the nucleotides comprising the polynucleotide can be RNA or DNA or a modified form of either type of nucleotide, such as a modified messenger RNA. Said modifications may include, but are not limited to, base modifications such as bromouridine, ribose modifications such as arabinoside and 2′,3′-dideoxyribose and internucleotide linkage modifications such as phosphorothioate, phosphorodithioate, phosphoroselenoate, phosphorodiselenoate, phosphoroanilothioate, phoshoraniladate and phosphoroamidate. The term “polynucleotide” specifically includes single and double stranded forms of DNA.

As used herein, a “polypeptide” or “protein” refers to a single, linear, and contiguous arrangement of covalently linked amino acids. Polypeptides can form one or more intrachain disulfide bonds. The terms polypeptide and protein also encompass embodiments where two polypeptide chains link together in a non-linear fashion, such as via an interchain disulfide bond. Herein, a protein or polypeptide may be an antibody or an antigen-binding fragment of an antibody.

As used herein, the term “transformation,” “transfection,” and “transduction” refer to the transfer of a polynucleotide into a cell. As used herein, the term “genetic transformation” refers to the transfer and incorporation of DNA, especially recombinant DNA, into a cell. The transferred nucleic acid can be introduced into a cell via an expression vector.

As used herein, the terms “treatment,” “treating,” or “ameliorating” refers to either a therapeutic treatment or prophylactic/preventative treatment. A treatment is therapeutic if at least one symptom of disease in an individual receiving treatment improves or a treatment can delay worsening of a progressive disease in an individual or prevent onset of additional associated diseases.

As used herein, an “epitope” refers to the amino acids conventionally bound by an immunoglobulin VH/VL pair, such as the antibodies and binding agents described herein. An epitope can be formed on a polypeptide from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a unique spatial conformation. An epitope defines the minimum binding site for an antibody, and thus represent the target of specificity of an antibody.

As used herein, “specifically binds” refers to the ability of an antibody described herein to bind to a target, such as MIC, with a KD 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. Specific binding can be influenced by, for example, the affinity and avidity of the binding agent and the concentration of target polypeptide. The person of ordinary skill in the art can determine appropriate conditions under which the antibodies and other binding agents described herein selectively bind to MIC using any suitable methods, such as titration of a binding agent in a suitable cell binding assay. A binding agent specifically bound to MIC is not displaced by a non-similar competitor. In certain embodiments, an anti-MIC antibody is said to specifically bind to MIC when it preferentially recognizes its target antigen, MIC, in a complex mixture of proteins and/or macromolecules.

In some embodiments, an anti-MIC antibody as described herein specifically binds to a MIC polypeptide with a dissociation constant (KD) of 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. In some embodiments, an anti-MIC antibody as described herein specifically binds to a MIC polypeptide with a dissociation constant (KD) of from about 10⁻⁵ M to 10⁻⁶ M. In some embodiments, an anti-MIC antibody as described herein specifically binds to a MIC polypeptide with a dissociation constant (KD) of from about 10⁻⁶ M to 10⁻⁷ M. In some embodiments, an anti-MIC antibody as described herein specifically binds to a MIC polypeptide with a dissociation constant (KD) of from about 10⁻⁷ M to 10⁻⁸ M. In some embodiments, an anti-MIC antibody as described herein specifically binds to a MIC polypeptide with a dissociation constant (KD) of from about 10⁻⁸ M to 10⁻⁹ M. In some embodiments, an anti-MIC antibody as described herein specifically binds to a MIC polypeptide with a dissociation constant (KD) from about 10⁻⁹ M to 10⁻¹⁰ M. In some embodiments, an anti-MIC antibody as described herein specifically binds to a MIC polypeptide with a dissociation constant (KD) of from about 10⁻¹⁰ M to 10⁻¹¹ M. In some embodiments, an anti-MIC antibody as described herein specifically binds to a MIC polypeptide with a dissociation constant (KD) of from about 10⁻¹¹ M to 10⁻¹² M. In some embodiments, an anti-MIC antibody as described herein specifically binds to a MIC polypeptide with a dissociation constant (KD) of less than 10⁻¹² M. In some embodiments, an anti-MIC antibody as described herein specifically binds to a MIC polypeptide with a dissociation constant (KD) of from about 1⁻⁹ M to 1.5⁻⁹ M.

As used herein, the term “consisting essentially of” refers to those elements required for a given embodiment. The term permits the presence of elements that do not materially affect the basic and novel or functional characteristic(s) of that embodiment.

The term “consisting of” refers to compositions, methods, and respective components thereof as described herein, which are exclusive of any element not recited in that description of the embodiment.

Other than in the examples, or where otherwise indicated, all numbers expressing quantities of ingredients or reaction conditions used herein should be understood as modified in all instances by the term “about.” The term “about” when used in connection with percentages can mean +/−1%.

The term “statistically significant” or “significantly” refers to statistical significance and generally means a two standard deviation (2SD) difference, above or below a reference value.

Other terms are defined herein within the description of the various aspects of the invention.

### Anti-MIC Antibodies

Provided herein are anti-MIC antibodies that specifically bind to MIC and comprise one or more point mutations in the Fc domain that reduce one or more Fc effector functions.

Major Histocompatibility Complex class I chain-related (MIC) polypeptides are cell surface transmembrane proteins. MIC polypeptides include, but are not limited to, human MICA isoforms (e.g. isoform 1, NCBI Ref Seq NP_000238.1 and 001170990) and other MICA isoforms), and human MICB isoforms (e.g. isoform 1, NCBI Ref Seq: NP_005922.2 and other MICB isoforms). In some embodiments, a MIC polypeptide refers to MICA. In some embodiments, a MIC polypeptide refers to MICB. In some embodiments, a MIC polypeptide refers to the shared structural features of MICA and MICB, i.e., the epitope(s) shared by MICA and MICB.

As used herein, “soluble MIC” or “sMIC” refers to a portion of a MIC polypeptide (MICA or MICB) containing the alpha1 and alpha 2 domains and the alpha3 domain or a portion of the alpha 3 domain up to the proteolytic cleavage site and lacking the transmembrane domain (e.g., an extracellular portion of MIC). In some embodiments, soluble MICA can comprise the amino acid sequence set forth in Genbank Accession No. CAA77031.1 (SEQ ID NO: 11), or a variant thereof, such as the amino acid sequences set forth in amino acid residues 24 to 297 of Genbank Accession No. AAU95072.1, AA045822.1, AFR69318.1, AFR69319.1 or AAH16929.1 or Ref Seq. NP_000238.1 or amino acids 1 to 274 of Genbank Accession No. CAE45581.1, or amino acids 1 to 273 of Genbank Accession No. AAD52069.1, AAD52070.1, or QDW65494.1 (the disclosures of which are incorporated by reference herein). In some embodiments, soluble MICB can comprise the amino acid sequence set forth in Genbank Accession No.: ARB08539.1 (SEQ ID NO: 12), AAB71646.1, AAB71647.1 or AAB71644.1, or a variant thereof, such as the amino acid sequences set forth in amino acid residues 24 to 297 of Genbank Accession No. ABO16470.1, ABB51802.1, AAB42011.1, AAB71643.1, or Q29980.1, or Ref SEQ No. NP_005922.2 or amino acid 1 to 273 of Genbank Accession No. AAC39848.1 AEK67483.1, AFR7773.1, AXY93666.1, CAB72098.1, or AAC39849.1 (the disclosures of which are incorporated by reference herein). Unless otherwise noted, use of the term “MIC” is intended to refer to both cell membrane-bound and soluble forms of MIC.

The term “exosomal MIC” is used to MIC protein that is present on an extracellular vesicle or exosome. The term MIC+ extracellular vesicle (MIC+ EV) is used herein to refer to extracellular vesicles comprising MIC.

The anti-MIC antibodies described herein comprise the antigen-binding domain sequences described in PCT/US2021/040445, which is incorporated herein by reference in its entirety. As used herein, an “antigen-binding domain” of an anti-MIC antibody refers to the VH and VL sequences of the anti-MIC antibody (set forth in SEQ ID NO: 7 and SEQ ID NO: 8). The amino acid sequences of the VH CDRs of the anti-MIC antibody are set forth in SEQ ID NO: 7 at amino acids 26-34 (GYSITSDYA, HCDR1, SEQ ID NO: 1), 50-58 (GYISYSGST, HCDR2, SEQ ID NO: 2) and 97-105 (ARGGTYFDY, HCDR3, SEQ ID NO: 3). The amino acid sequences of the VL CDRs of the anti-MIC antibody are set forth in SEQ ID NO: 8 at amino acids 24-32 (RASAHINNW, LCDR1, SEQ ID NO: 4), 50-56 (DATSLES, LCDR2, SEQ ID NO: 5) and 98-107 (QHYWSTPWT, LCDR3, SEQ ID NO: 6). The phrase “wherein the CDRs of the heavy or light chain variable regions are not modified” refers to these VH and VL CDRs (SEQ ID NOs: 1-6), which do not have amino acid substitutions, deletions, or insertions.

In some embodiments, the anti-MIC antibody described herein comprises a heavy chain variable (VH) region and a light chain variable (VL) region, wherein the VH region comprises a complementarity determining region HCDR1 having the amino acid sequence set forth in SEQ ID NO: 1, a HCDR2 having the amino acid sequence set forth in SEQ ID NO: 2 and a HCDR3 having the amino acid sequence set forth in SEQ ID NO: 3, and the VL region comprises a LCDR1 having the amino acid sequence set forth in SEQ ID NO: 4, a LCDR2 having the amino acid sequence set forth in SEQ ID NO: 5, and a LCDR3 having the amino acid sequence set forth in SEQ ID NO: 6, and wherein each VH and VL comprises a humanized framework region. In some embodiments, the VH framework region is derived from a human germline gene having the amino acid sequence set forth in IMGT IGHV4-59*11 (SEQ ID NO: 13) and IGHJ4*01 (SEQ ID NO: 14) or IGHV4-30-4*01 (SEQ ID NO: 15) and IGHJ4*01 (SEQ ID NO:14). In some embodiments, the VL framework region is derived from a human germline gene having the amino acid sequence set forth in IMGT IGKV1-NL1*01 (SEQ ID NO: 16) and IMGT IGKJ1*01 (SEQ ID NO: 17), IMGT IGKV1-33*01 (SEQ ID NO: 18) and IMGT IGKJ1*01 (SEQ ID NO: 17) or IMGT IGKV1-5*01 (SEQ ID NO: 19) and IMGT IGKJ1*01 (SEQ ID NO: 17).

In some embodiments, the anti-MIC antibody described herein comprises (i) a heavy chain variable region having the amino acid sequence set forth in SEQ ID NO: 7, and (ii) a light chain variable region having the amino acid sequence set forth in SEQ ID NO: 8. In some embodiments, the anti-MIC antibody described herein comprises (i) a heavy chain variable region having the amino acid sequence set forth in SEQ ID NO: 7 and (ii) a light chain variable region having the amino acid sequence set forth in SEQ ID NO: 8, wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the anti-MIC antibody described herein comprises (i) a heavy chain variable region having the amino acid sequence set forth in SEQ ID NO: 7 and (ii) a light chain variable region having the amino acid sequence set forth in SEQ ID NO: 8, wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

As to the VH and VL amino acid sequences, one of skill will recognize that individual substitutions, deletions or additions (insertions) to a nucleic acid encoding the VH or VL, or amino acids in polypeptide that alter a single amino acid or a small percentage of amino acids in the encoded sequence is a “conservatively modified variant”, where the alteration results in the substitution of an amino acid with a chemically similar amino acid (a conservative amino acid substitution) and the altered polypeptide retains the ability to specifically bind to MIC.

In some embodiments, a conservatively modified variant of an anti-MIC antibody or can have alterations in the FR (i.e., other than in the CDRs), e.g. a conservatively modified variant of an anti-MIC antibody has the amino acid sequences of the VH and VL CDRs (set forth in SEQ ID NOs: 11-16) and has at least one conservative amino acid substitution in the FR. In some embodiments, the VH and VL amino acid sequences (set forth in SEQ ID NOs: 1 and 2, respectively) collectively have no more than 8 or 6 or 4 or 2 or 1 conservative amino acid substitutions in the FR, as compared to the amino acid sequences of the VH and VL (SEQ ID Nos: 1 and 2, respectively). In some embodiments, the VH and VL amino acid sequences (set forth in SEQ ID NOs: 1 and 2, respectively) have 8 to 1, 6 to 1, 4 to 1 or 2 to 1 conservative amino acid substitutions in the FR, as compared to the amino acid sequences of the VH and VL (set forth in SEQ ID Nos: 1 and 2, respectively).

For conservative amino acid substitutions, a given amino acid can be replaced by a residue having similar physiochemical characteristics, e.g., substituting one aliphatic residue for another (such as lie, Val, Leu, or Ala for one another), or substitution of one polar residue for another (such as between Lys and Arg; Glu and Asp; or Gln and Asn). Other such conservative amino acid substitutions, e.g., substitutions of entire regions having similar hydrophobicity characteristics, are well known. Polypeptides comprising conservative amino acid substitutions can be tested in any one of the assays described herein to confirm that a desired activity, e.g. antigen-binding activity and specificity of a native or reference polypeptide is retained, i.e., to MIC (sMIC and/or membrane bound MIC).

For conservative substitutions, amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His (H).

Alternatively, for conservative substitutions naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe. Non-conservative substitutions will entail exchanging a member of one of these classes or another class.

Particular conservative substitutions include, for example; Ala to Gly or to Ser; Arg to Lys; Asn to Gln or to His; Asp to Glu; Cys to Ser; Gln to Asn; Glu to Asp; Gly to Ala or to Pro; His to Asn or to Gln; Ile to Leu or to Val; Leu to Ile or to Val; Lys to Arg, to Gln or to Glu; Met to Leu, to Tyr or to Ile; Phe to Met, to Leu or to Tyr; Ser to Thr; Thr to Ser; Trp to Tyr; Tyr to Trp; and/or Phe to Val, to lie or to Leu.

In some embodiments, a conservatively modified variant on an anti-MIC antibody preferably is at least 90%, at least 91%, at least 92%, at least 93%, at least 94%, at least 95%, at least 96%, at least 97%, at least 98%, at least 99%, or more, identical to the reference VH or VL sequence, wherein the VH and VL CDRs (SEQ ID NOs:11-16) are not modified. The degree of homology (percent identity) between the reference and modified sequence can be determined, for example, by comparing the two sequences using freely available computer programs commonly employed for this purpose on the world wide web (e.g. BLASTp or BLASTn with default settings).

In some embodiments, the VH and VL amino acid sequences (set forth in SEQ ID NOs:1 and 2, respectively) collectively have no more than 8 or 6 or 4 or 2 or 1 conservative amino acid substitutions in the framework regions, as compared to the amino acid sequences of the VH and VL (set forth in SEQ ID NOs: 1 and 2, respectively). In some embodiments, the VH and VL amino acid sequences (set forth in SEQ ID Nos: 1 and 2, respectively) collectively have 8 to 1, or 6 to 1, or 4 to 1, or 2 to 1 conservative amino acid substitutions in the framework regions, as compared to the amino acid sequences of the VH and VL (set forth in SEQ ID NOs: 1 and 2, respectively). In some embodiments, the VH and VL amino acid sequences (set forth in SEQ ID NOs: 1 and 2, respectively) collectively have no more than 8 or 6 or 4 or 2 or 1 amino acid substitutions, deletions or insertions in the framework regions, as compared to the amino acid sequences of the VH and VL (set forth in SEQ ID NOs: 1 and 2, respectively). In some embodiments, the VH and VL amino acid sequences (set forth in SEQ ID NOs: 1 and 2, respectively) have 8 to 1, 6 to 1, 4 to 1, or 2 to 1 conservative amino acid substitutions in the framework regions, as compared to the amino acid sequences of the VH and VL (set forth in SEQ ID NOs: 1 and 2, respectively). In some embodiments, the VH and VL amino acid sequences (set forth in SEQ ID NOs:1 and 2, respectively) collectively have no more than 8 or 6 or 4 or 2 or 1 amino acid substitutions, deletions or insertions, as compared to the amino acid sequences of the VH and VL (set forth in SEQ ID NOs:1 and 2, respectively).

### Fc Domain Mutations

The anti-MIC antibodies described herein comprise one or more point mutations in the Fc domain that reduce one or more Fc effector functions. The anti-MIC antibodies demonstrate superior biological effects compared to previously described anti-MIC antibodies comprising the same antigen-binding domain sequences but not comprising the one or more point mutations in the Fc domain.

As used herein, “Fc domain” describes the constant region of an antibody that can bind to or be bound by an Fc receptor (FcR) and determines the isotype of the antibody. Generally, an Fc domain comprises the CH2 and CH3 domains of an antibody heavy chain, optionally comprising a CH4 domain based on the isotype of the antibody, and is capable of binding one or more Fc receptors. FcRs are organized into classes (e.g., gamma (γ), alpha (a) and epsilon (s)) based on the class of antibody that the FcR recognizes. The FcaR class can bind to IgA and includes several isoforms, such as FcaRI (CD89). The FcγR class can bind to IgG and includes several isoforms, FcγRI (CD64), FcγRIIA (CD32a), FcγRIIB (CD32b), FcγRIIIA (CD16a), and FcγRIIIB (CD16b). FcγRIIIA (CD16a) has two major variants, F158 or V158.

The sequences of antibody Fc domains are known in the art and the amino acids within the Fc domains are numbered according to one of several accepted numbering systems. Unless otherwise indicated herein, reference to a particular amino acid position in the Fc domain of an antibody is based on the EU index as in Kabat et al., Sequences of Proteins of Immunological Interest, 5th Ed. Public Health Service, National Institutes of Health, Bethesda, MD (1991).

In some embodiments, the anti-MIC antibody is an IgG1 isotype. In some embodiments, the anti-MIC antibody is an IgG2 isotype. In some embodiments, the anti-MIC antibody is an IgG3 isotype. In some embodiments, the anti-MIC antibody is an IgG4 isotype.

Modification of a native (or reference) amino acid sequence can be accomplished by any of a number of techniques known to one of skill in the art. Mutations can be introduced, for example, at particular loci by synthesizing oligonucleotides containing the desired mutant sequence, flanked by restriction sites enabling ligation to fragments of the native sequence. Following ligation, the resulting reconstructed sequence encodes a variant having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion desired. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties.

The anti-MIC antibodies described herein comprise an Fc domain comprising the mutations P329G, L234A, and L235A, referred to herein as the PG-LALA mutations. These mutations have been previously described to reduce Fc binding to complement components, FcγRs, and to reduce complement-dependent cytotoxicity (See Lo et al., J Bio Chem, 292:9(2017); 3900-3908), thereby resulting in an “effectorless” Fc domain. However, these point mutations result in unexpected results in the context of the anti-MIC antibodies described herein.

Previously published data with an effectorless variant of the anti-MIC antibody 6E1, in which an N297G mutation was introduced into the Fc domain, indicate that the therapeutic effect of the anti-MIC antibody is dependent on Fc cross-linking in order to trigger NK cell activation. This study demonstrated that soluble MICA formed complexes with the 6E1 antibody and that these immune complexes reversed the immunosuppressive activities of soluble MICA by activating NKG2D through a Fc receptor-dependent mechanism. Pre-formed anti-MICA immune complexes containing wild-type Fc domain induced IFN-γ and TNF-α secretion by NK cells in the absence of tumor cells. In contrast, immune complexes formed with an effectorless Fc mutant of 6E1 (N297G) did not, suggesting that the immunostimulatory capabilities of the anti-MIC antibody was dependent on Fc receptor crosslinking.

It was therefore expected that introduction of the P329G, L234A, and L235A into the Fc domain of the anti-MIC antibodies described herein would similarly reduce the immunostimulatory capacity of the antibodies. Surprisingly, anti-MIC antibodies comprising the PG-LALA mutations were able to reverse immunosuppression induced by MIC+ extracellular vesicles. See Examples 2-6. Therefore, the PG-LALA mutation enhanced the therapeutic efficacy of the anti-MIC antibodies described herein, in contrast to what was suggested by previously published studies.

Therefore, in some embodiments, the present disclosure provides an anti-MIC antibody comprising an Fc domain comprising the point mutations P329G, L234A, and L235A. Therefore, in some embodiments, the present disclosure provides an anti-MIC antibody comprising an Fc domain comprising the point mutations L234A and L235A. In some embodiments, an anti-MIC antibody heavy chain is of the IgG1 isotype and has the amino acid sequence set forth in SEQ ID NO:9, further comprising the amino acid mutations at L234A and L235A. In some embodiments, an anti-MIC antibody heavy chain is of the IgG1 isotype and has the amino acid sequence set forth in SEQ ID NO:9, further comprising the amino acid mutations at P329G, L234A, and L235A. In some embodiments, an anti-MIC antibody light chain is of the kappa isotype and has the amino acid sequence set forth in SEQ ID NO: 10.

In some embodiments, an anti-MIC antibody has fully human constant regions. In some embodiments, an anti-MIC antibody has non-human constant regions. In some embodiments, an anti-MIC antibody heavy chain is of the IgG1 isotype and has the amino acid sequence set forth in SEQ ID NO: 9, further comprising the amino acid mutations at L234A and L235A. In some embodiments, an anti-MIC antibody heavy chain is of the IgG1 isotype and has the amino acid sequence set forth in SEQ ID NO:9, further comprising the amino acid mutations at P329G, L234A, and L235A. In some embodiments, an anti-MIC antibody light chain is of the kappa isotype and has the amino acid sequence set forth in SEQ ID NO: 10.

The anti-MIC antibody provide herein comprises an Fc domain having a sequence that has been modified, as compared to a wild type or reference sequence, to comprise one or more point mutations that decrease an effector function of the Fc domain, e.g., reduced binding to one or more Fc receptors (FcRs), compared to an anti-MIC antibody with a wildtype or reference Fc domain. Such a modification(s) can alter the ability of the antibody or antigen binding portion to interact with immune cells. Such a modification or series of modifications to an Fc domain or region can permit selective binding of an Fc domain to FcRs on immune cells, or can reduce or eliminate the interaction of an antibody or antigen binding portion having the modified domain with immune cells. For example, modifications to an Fc domain can reduce binding of an Fc domain to Fc gamma receptors, but retain the ability of the Fc domain to bind to FcRn.

In some embodiments, the Fc domain of the anti-MIC antibodies provided herein exhibit reduced binding affinity to one or more Fc receptors. In some embodiments, the Fc domain of the anti-MIC antibodies provided herein exhibit reduced binding affinity to one or more Fcγ receptors. In some embodiments, an Fc domain or region can exhibit reduced binding affinity to FcRn receptors. In some embodiments, an Fc domain or region can exhibit reduced binding affinity to Fcγ and to FcRn receptors. In some embodiments, an Fc domain is an Fc null domain. As used herein, an “Fc null” or an “effectorless Fc” refers to a domain that exhibits weak to no binding to any of the Fcγ receptors. In some embodiments, an Fc null domain or region exhibits a reduction in binding affinity (e.g., increase in Kd) to Fcγ receptors of at least 1000-fold.

In certain embodiments, an Fc domain has decreased binding affinity for one or more of FcγRI (CD64), FcγRIIA (CD32), FcγRIIIA (CD16a), FcγRIIIB (CD16b), or any combination thereof. In order to decrease binding affinity of an Fc domain or region to an Fc receptor, the Fc domain or region may comprise one or more amino acid substitutions that reduce the binding affinity of the Fc domain or region to an Fc receptor.

In some embodiments, the compositions and methods described herein relate to inhibition of the immune suppressive effects of exosomal MIC. In some embodiments, inhibition of exosomal MIC increases NKG2D expression on NK cells, increases NK cell degranulation, and/or increases NK cell cytolysis of target tumor cells.

### Antibody Production

In various embodiments, MIC antibodies, antigen binding portions thereof and other binding agents can be produced in human, murine or other animal-derived cells lines. Recombinant DNA expression can be used to produce MIC antibodies, antigen binding portions thereof and other binding agents. This allows the production of MIC antibodies as well as a spectrum of MIC antigen binding portions and other binding agents (including fusion proteins) in a host species of choice. The production of anti-MIC antibodies in bacteria, yeast, transgenic animals and chicken eggs are also alternatives for cell-based production systems. The main advantages of transgenic animals are potential high yields from renewable sources.

In some embodiments, a MIC VH polypeptide having the amino acid sequence set forth in SEQ ID NO: 7 is encoded by a nucleic acid. In some embodiments, a MIC VL polypeptide having the amino acid sequence set forth in SEQ ID NO: 8 is encoded by a nucleic acid. In some embodiments, a MIC VH polypeptide having the amino acid sequence set forth in SEQ ID NO: 7 is encoded by a nucleic acid having the sequence set forth in SEQ ID NO: 20. In some embodiments, a MIC VL polypeptide having the amino acid sequence set forth in SEQ ID NO: 8 is encoded by a nucleic acid having the sequence set forth in SEQ ID NO: 21.

As used herein, the term “nucleic acid” or “nucleic acid sequence” or “polynucleotide sequence” or “nucleotide” refers to a polymeric molecule incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. In some embodiments, the nucleic acid can be a cDNA, e.g., a nucleic acid lacking introns.

Nucleic acid molecules encoding the amino acid sequence of an anti-MIC antibody can be prepared by a variety of methods known in the art. These methods include, but are not limited to, preparation of synthetic nucleotide sequences encoding an anti-MIC antibody, antigen binding portion or other binding agent(s). In addition, oligonucleotide-mediated (or site-directed) mutagenesis, PCR-mediated mutagenesis, and cassette mutagenesis can be used to prepare nucleotide sequences encoding an anti-MIC antibody or antigen binding portion as well as other binding agents. A nucleic acid sequence encoding at least an anti-MIC antibody or a polypeptide thereof, as described herein, can be recombined with vector DNA in accordance with conventional techniques, such as, for example, blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases. Techniques for such manipulations are disclosed, e.g., by Maniatis et al., Molecular Cloning, Lab. Manual (Cold Spring Harbor Lab. Press, NY, 1982 and 1989), and Ausubel et al., Current Protocols in Molecular Biology (John Wiley & Sons), 1987-1993, and can be used to construct nucleic acid sequences and vectors that encode an anti-MIC antibody.

A nucleic acid molecule, such as DNA, is said to be “capable of expressing” a polypeptide if it contains nucleotide sequences that contain transcriptional and translational regulatory information and such sequences are “operably linked” to nucleotide sequences that encode the polypeptide. An operable linkage is a linkage in which the regulatory DNA sequences and the DNA sequence sought to be expressed (e.g., an anti-MIC antibody) are connected in such a way as to permit gene expression of a polypeptide(s) or antigen binding portions in recoverable amounts. The precise nature of the regulatory regions needed for gene expression may vary from organism to organism, as is well known in the analogous art. See, e.g., Sambrook et al., 1989; Ausubel et al., 1987-1993.

Accordingly, the expression of an anti-MIC antibody as described herein can occur in either prokaryotic or eukaryotic cells. Suitable hosts include bacterial or eukaryotic hosts, including yeast, insects, fungi, bird and mammalian cells either in vivo, or in situ, or host cells of mammalian, insect, bird or yeast origin. The mammalian cell or tissue can be of human, primate, hamster, rabbit, rodent, cow, pig, sheep, horse, goat, dog or cat origin, but any other mammalian cell may be used. Further, by use of, for example, the yeast ubiquitin hydrolase system, in vivo synthesis of ubiquitin-transmembrane polypeptide fusion proteins can be accomplished. The fusion proteins so produced can be processed in vivo or purified and processed in vitro, allowing synthesis of an anti-MIC antibody or as described herein with a specified amino terminus sequence. Moreover, problems associated with retention of initiation codon-derived methionine residues in direct yeast (or bacterial) expression maybe avoided. (See, e.g., Sabin et al., 7 Bio/Technol. 705 (1989); Miller et al., 7 Bio/Technol. 698 (1989).) Any of a series of yeast gene expression systems incorporating promoter and termination elements from the actively expressed genes coding for glycolytic enzymes produced in large quantities when yeast are grown in medium rich in glucose can be utilized to obtain recombinant MIC antibodies or antigen-binding portions thereof. Known glycolytic genes can also provide very efficient transcriptional control signals. For example, the promoter and terminator signals of the phosphoglycerate kinase gene can be utilized.

Production of anti-MIC antibodies in insects can be achieved, for example, by infecting an insect host with a baculovirus engineered to express a polypeptide by methods known to those of ordinary skill in the art. See Ausubel et al., 1987-1993.

In some embodiments, the introduced nucleic acid sequence (encoding an anti-MIC antibody described herein) is incorporated into a plasmid or viral vector capable of autonomous replication in a recipient host cell. Any of a wide variety of vectors can be employed for this purpose and are known and available to those of ordinary skill in the art. See, e.g., Ausubel et al., 1987-1993. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Exemplary prokaryotic vectors known in the art include plasmids such as those capable of replication in E. coli. Other gene expression elements useful for the expression of DNA encoding MIC antibodies or antigen-binding portions thereof include, but are not limited to (a) viral transcription promoters and their enhancer elements, such as the SV40 early promoter. (Okayama et al., 3 Mol. Cell. Biol. 280 (1983)), Rous sarcoma virus LTR (Gorman et al., 79 PNAS 6777 (1982)), and Moloney murine leukemia virus LTR (Grosschedl et al., 41 Cell 885 (1985)); (b) splice regions and polyadenylation sites such as those derived from the SV40 late region (Okayarea et al., 1983), and (c) polyadenylation sites such as in SV40 (Okayama et al., 1983). Immunoglobulin-encoding DNA genes can be expressed as described by Liu et al., infra, and Weidle et al., 51 Gene 21 (1987), using as expression elements the SV40 early promoter and its enhancer, the mouse immunoglobulin H chain promoter enhancers, SV40 late region mRNA splicing, rabbit S-globin intervening sequence, immunoglobulin and rabbit S-globin polyadenylation sites, and SV40 polyadenylation elements.

For immunoglobulin encoding nucleotide sequences, the transcriptional promoter can be, for example, human cytomegalovirus, the promoter enhancers can be cytomegalovirus and mouse/human immunoglobulin.

In some embodiments, for expression of DNA coding regions in rodent cells, the transcriptional promoter can be a viral LTR sequence, the transcriptional promoter enhancers can be either or both the mouse immunoglobulin heavy chain enhancer and the viral LTR enhancer, and the polyadenylation and transcription termination regions. In other embodiments, DNA sequences encoding other proteins are combined with the above-recited expression elements to achieve expression of the proteins in mammalian cells.

Each coding region or gene fusion is assembled in, or inserted into, an expression vector. Recipient cells capable of expressing the MIC variable region(s) or antigen binding portions thereof (e.g., a VH having the amino acid sequence set forth in SEQ ID NO: 7 and/or a VL having the amino acid sequence set forth in SEQ ID NO: 8 or a variant thereof as described herein) are then transfected singly with nucleotides encoding an anti-MIC antibody or an antibody polypeptide or antigen-binding portion thereof, or are co-transfected with a polynucleotide(s) encoding VH and a VL chain coding regions. The transfected recipient cells are cultured under conditions that permit expression of the incorporated coding regions and the expressed antibody chains or intact antibodies or antigen binding portions are recovered from the culture.

In some embodiments, the nucleic acids containing the coding regions encoding an anti-MIC antibody (e.g., a VH having the amino acid sequence set forth in SEQ ID NO: 7 and/or a VL having the amino acid sequence set forth in SEQ ID NO: 8 or a variant thereof as described herein) are assembled in separate expression vectors that are then used to co-transfect a recipient host cell. Each vector can contain one or more selectable genes. For example, in some embodiments, two selectable genes are used, a first selectable gene designed for selection in a bacterial system and a second selectable gene designed for selection in a eukaryotic system, wherein each vector has a set of coding regions. This strategy results in vectors which first direct the production, and permit amplification, of the nucleotide sequences in a bacterial system. The DNA vectors so produced and amplified in a bacterial host are subsequently used to co-transfect a eukaryotic cell, and allow selection of a co-transfected cell carrying the desired transfected nucleic acids (e.g., containing MIC antibody heavy and light chains). Non-limiting examples of selectable genes for use in a bacterial system are the gene that confers resistance to ampicillin and the gene that confers resistance to chloramphenicol. Selectable genes for use in eukaryotic transfectants include the xanthine guanine phosphoribosyl transferase gene (designated gpt) and the phosphotransferase gene from Tn5 (designated neo). Alternatively, the fused nucleotide sequences encoding VH and VL chains can be assembled on the same expression vector.

For transfection of the expression vectors and production of the MIC antibodies or antigen binding portions thereof, the recipient cell line can be a Chinese Hamster ovary cell line (e.g., DG44) or a myeloma cell. Myeloma cells can synthesize, assemble and secrete immunoglobulins encoded by transfected immunoglobulin genes and possess the mechanism for glycosylation of the immunoglobulin. For example, in some embodiments, the recipient cell is the recombinant Ig-producing myeloma cell SP2/0 (ATCC #CRL 8287). SP2/0 cells only produce immunoglobulins encoded by the transfected genes. Myeloma cells can be grown in culture or in the peritoneal cavity of a mouse, where secreted immunoglobulin can be obtained from ascites fluid.

An expression vector encoding an anti-MIC antibody (e.g., a VH having the amino acid sequence set forth in SEQ ID NO: 7 and/or a VL having the amino acid sequence set forth in SEQ ID NO: 8 or a variant thereof as described herein) can be introduced into an appropriate host cell by any of a variety of suitable means, including such biochemical means as transformation, transfection, protoplast fusion, calcium phosphate-precipitation, and application with polycations such as diethylaminoethyl (DEAE) dextran, and such mechanical means as electroporation, direct microinjection and microprojectile bombardment. Johnston et al., 240 Science 1538 (1988), as known to one of ordinary skill in the art.

Yeast provides certain advantages over bacteria for the production of immunoglobulin heavy and light chains. Yeasts carry out post-translational peptide modifications including glycosylation. A number of recombinant DNA strategies exist that utilize strong promoter sequences and high copy number plasmids which can be used for production of the desired proteins in yeast. Yeast recognizes leader sequences of cloned mammalian gene products and secretes polypeptides bearing leader sequences (i.e., pre-polypeptides). See, e.g., Hitzman et al., 11th Intl. Conf. Yeast, Genetics & Molec. Biol. (Montpelier, France, 1982).

Yeast gene expression systems can be routinely evaluated for the levels of production, secretion and the stability of antibodies, and assembled MIC antibodies and antigen binding portions thereof. Various yeast gene expression systems incorporating promoter and termination elements from the actively expressed genes coding for glycolytic enzymes produced in large quantities when yeasts are grown in media rich in glucose can be utilized. Known glycolytic genes can also provide very efficient transcription control signals. For example, the promoter and terminator signals of the phosphoglycerate kinase (PGK) gene can be utilized. Another example is the translational elongation factor 1alpha promoter. A number of approaches can be taken for evaluating optimal expression plasmids for the expression of immunoglobulins in yeast. See II DNA Cloning 45, (Glover, ed., IRL Press, 1985) and e.g., U.S. Publication No. US 2006/0270045 A1.

Bacterial strains can also be utilized as hosts for the production of the antibody molecules or antigen binding portions thereof described herein, E. coli K12 strains such as E. coli W3110 (ATCC 27325), Bacillus species, enterobacteria such as Salmonella typhimurium or Serratia marcescens, and various Pseudomonas species can be used. Plasmid vectors containing replicon and control sequences which are derived from species compatible with a host cell are used in connection with these bacterial hosts. The vector carries a replication site, as well as specific genes which are capable of providing phenotypic selection in transformed cells. A number of approaches can be taken for evaluating the expression plasmids for the production of MIC antibodies and antigen binding portions thereof in bacteria (see Glover, 1985; Ausubel, 1987, 1993; Sambrook, 1989; Colligan, 1992-1996).

Host mammalian cells can be grown in vitro or in vivo. Mammalian cells provide post-translational modifications to immunoglobulin molecules including leader peptide removal, folding and assembly of VH and VL chains, glycosylation of the antibody molecules, and secretion of functional antibody and/or antigen binding portions thereof.

Mammalian cells which can be useful as hosts for the production of antibody proteins, in addition to the cells of lymphoid origin described above, include cells of fibroblast origin, such as Vero (ATCC CRL 81) or CHO-K1 (ATCC CRL 61) cells. Exemplary eukaryotic cells that can be used to express immunoglobulin polypeptides include, but are not limited to, COS cells, including COS 7 cells; 293 cells, including 293-6E cells; CHO cells, including CHO-S and DG44 cells; PERC6™ cells (Crucell); and NSO cells. In some embodiments, a particular eukaryotic host cell is selected based on its ability to make desired post-translational modifications to the heavy chains and/or light chains. For example, in some embodiments, CHO cells produce polypeptides that have a higher level of sialylation than the same polypeptide produced in 293 cells.

In some embodiments, one or more MIC antibodies or antigen-binding portions thereof (e.g., a VH having the amino acid sequence set forth in SEQ ID NO: 7 and/or a VL having the amino acid sequence set forth in SEQ ID NO: 8 or a variant thereof as described herein) can be produced in vivo in an animal that has been engineered or transfected with one or more nucleic acid molecules encoding the polypeptides, according to any suitable method.

In some embodiments, an antibody (e.g., a VH having the amino acid sequence set forth in SEQ ID NO: 7 and/or a VL having the amino acid sequence set forth in SEQ ID NO: 8 or a variant thereof as described herein) is produced in a cell-free system. Non-limiting exemplary cell-free systems are described, e.g., in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

Many vector systems are available for the expression of the VH and VL chains (e.g., a VH having the amino acid sequence set forth in SEQ ID NO: 7 and/or a VL having the amino acid sequence set forth in SEQ ID NO: 8 or a variant thereof as described herein) in mammalian cells (see Glover, 1985). Various approaches can be followed to obtain intact antibodies. As discussed above, it is possible to co-express VH and VL chains and optionally the associated constant regions in the same cells to achieve intracellular association and linkage of VH and VL chains into complete tetrameric H₂L2 antibodies or antigen-binding portions thereof. The co-expression can occur by using either the same or different plasmids in the same host. Nucleic acids encoding the VH and VL chains or antigen binding portions thereof (e.g., a VH having the amino acid sequence set forth in SEQ ID NO: 7 and a VL having the amino acid sequence set forth in SEQ ID NO: 8 or a variant thereof as described herein) can be placed into the same plasmid, which is then transfected into cells, thereby selecting directly for cells that express both chains. Alternatively, cells can be transfected first with a plasmid encoding one chain, for example the VL chain, followed by transfection of the resulting cell line with a VH chain plasmid containing a second selectable marker. Cell lines producing antibodies, antigen-binding portions thereof via either route could be transfected with plasmids encoding additional copies of peptides, VH, VL, or VH plus VL chains (e.g., a VH having the amino acid sequence set forth in SEQ ID NO: 7 and/or a VL having the amino acid sequence set forth in SEQ ID NO: 8 or a variant thereof as described herein) in conjunction with additional selectable markers to generate cell lines with enhanced properties, such as higher production of assembled MIC antibodies or antigen binding portions thereof or enhanced stability of the transfected cell lines.

Additionally, plants have emerged as a convenient, safe and economical alternative expression system for recombinant antibody production, which are based on large scale culture of microbes or animal cells. MIC binding antibodies or antigen binding portions can be expressed in plant cell culture, or plants grown conventionally. The expression in plants may be systemic, limited to sub-cellular plastids, or limited to seeds (endosperms). See, e.g., U.S. Patent Pub. No. 2003/0167531; U.S. Pat. Nos. 6,080,560; 6,512,162; WO 0129242. Several plant-derived antibodies have reached advanced stages of development, including clinical trials (see, e.g., Biolex, N.C.).

For intact antibodies, the variable regions (VH and VL) of the MIC antibodies (e.g., a VH having the amino acid sequence set forth in SEQ ID NO: 7 and/or a VL having the amino acid sequence set forth in SEQ ID NO: 8 or a variant thereof as described herein) are typically linked to at least a portion of an immunoglobulin constant region (Fc), typically that of a human immunoglobulin. Human constant region DNA sequences can be isolated in accordance with well-known procedures from a variety of human cells, such as immortalized B-cells (WO 87/02671; which is incorporated by reference herein in its entirety). An anti-MIC antibody can contain both light chain and heavy chain constant regions. The heavy chain constant region can include CH1, hinge, CH2, CH3, and, sometimes, CH4 regions.

Intact (e.g., whole) antibodies, their dimers, individual light and heavy chains, or antigen binding portions thereof can be recovered and purified by known techniques, e.g., immunoadsorption or immunoaffinity chromatography, chromatographic methods such as HPLC (high performance liquid chromatography), ammonium sulfate precipitation, gel electrophoresis, or any combination of these. See generally, Scopes, Protein Purification (Springer-Verlag, N.Y., 1982). Substantially pure MIC binding antibodies or antigen binding portions thereof of at least about 90% to 95% homogeneity are advantageous, as are those with 98% to 99% or more homogeneity, particularly for pharmaceutical uses. Once purified, partially or to homogeneity as desired, an intact anti-MIC antibody can then be used therapeutically or in developing and performing assay procedures, immunofluorescent staining, and the like. See generally, Vols. I & II Immunol. Meth. (Lefkovits & Pernis, eds., Acad. Press, NY, 1979 and 1981).

Additionally, and as described herein, an anti-MIC antibody can be further optimized to decrease potential immunogenicity, while maintaining functional activity, for therapy in humans. In some embodiments, an optimized MIC binding antibody is derived from an anti-MIC antibody comprising (i) a heavy chain variable region having the amino acid sequence set forth in SEQ ID NO: 7 and (ii) a light chain variable region having the amino acid sequence set forth in SEQ ID NO: 8, wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, an optimized MIC binding antibody is derived from an anti-MIC antibody comprising (i) a heavy chain variable region having the amino acid sequence set forth in SEQ ID NO: 7 and (ii) a light chain variable region having the amino acid sequence set forth in SEQ ID NO: 8, wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified. In this regard, functional activity means an anti-MIC antibody capable of displaying one or more known functional activities associated with an anti-MIC antibody comprising (i) a heavy chain variable region having the amino acid sequence set forth in SEQ ID NO: 7 and (ii) a light chain variable region having the amino acid sequence set forth in SEQ ID NO: 8. In any of these embodiments, the functional activity of the MIC binding antibody includes specifically binding to MIC with a binding affinity greater than that of antibody B10G5. Additional functional activities include inhibition of MIC, and/or anti-cancer activity. Additionally, an anti-MIC antibody having functional activity means the polypeptide exhibits activity similar to, or better than, the activity of a reference antibody as described herein (e.g., an anti-MIC antibody comprising (i) a heavy chain variable region having the amino acid sequence set forth in SEQ ID NO: 7 and (ii) a light chain variable region having the amino acid sequence set forth in SEQ ID NO: 8 or a variant thereof, as described herein), as measured in a particular assay, such as, for example, a biological assay, with or without dose dependency. In the case where dose dependency does exist, it need not be identical to that of the reference antibody or antigen-binding portion thereof, but rather substantially similar to or better than the dose-dependence in a given activity as compared to the reference antibody as described herein (i.e., the candidate polypeptide will exhibit greater activity relative to the reference antibody).

Other aspects of the anti-MIC antibodies relate to compositions comprising active ingredients (i.e., including an anti-MIC antibody as described herein or a nucleic acid encoding an antibody as described herein). In some embodiments, the composition is a pharmaceutical composition. As used herein, the term “pharmaceutical composition” refers to the active agent in combination with a pharmaceutically acceptable carrier accepted for use in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The preparation of a pharmacological composition that contains active ingredients dissolved or dispersed therein is well understood in the art and need not be limited based on any particular formulation. Typically, such compositions are prepared as injectable either as liquid solutions or suspensions; however, solid forms suitable for rehydration, or suspensions, in liquid prior to use can also be prepared. A preparation can also be emulsified or presented as a liposome composition. An anti-MIC antibody can be mixed with excipients that are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, a pharmaceutical composition can contain minor amounts of auxiliary substances such as wetting or emulsifying agents, pH buffering agents and the like which enhance or maintain the effectiveness of the active ingredient (e.g., an anti-MIC antibody). The pharmaceutical compositions as described herein can include pharmaceutically acceptable salts of the components therein. Pharmaceutically acceptable salts include the acid addition salts (formed with the free amino groups of a polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain the active ingredients (e.g., an anti-MIC antibody and/or antigen binding portions thereof) and water, and may contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent that will be effective in the treatment of a particular disorder or condition will depend on the nature of the disorder or condition, and can be determined by standard clinical techniques.

In some embodiments, a pharmaceutical composition comprising an anti-MIC antibody as described herein or a nucleic acid encoding an anti-MIC antibody as described herein can be a lyophilisate.

In some embodiments, a syringe comprising a therapeutically effective amount of an anti-MIC antibody or a pharmaceutical composition thereof described herein is provided.

### Methods of Use

In some embodiments, the subject is in need of treatment for a cancer and/or a malignancy. In some embodiments, the subject is in need of treatment for a MIC+ cancer or a MIC+ malignancy, such as for example an epithelial cell cancer, a MIC+ solid tumor or a MIC+ hematopoietic malignancy. In some embodiments, the method is for treating a subject having a MIC+ cancer or malignancy. In some embodiments, the method is for treating an epithelial cell cancer or a hematopoietic malignancy in a subject. In some embodiments, the method is for treating a MIC+ epithelial cell cancer or a MIC+ hematopoietic malignancy in a subject. As used herein, “epithelial cell cancer” refers to a cancer that develops from epithelial cells.

The methods described herein include administering a therapeutically effective amount of an anti-MIC antibody. As used herein, the phrase “therapeutically effective amount”, “effective amount” or “effective dose” refers to an amount of the anti-MIC antibody as described herein that provides a therapeutic benefit in the treatment of, management of or prevention of relapse of a tumor or malignancy, e.g. an amount that provides a statistically significant decrease in at least one symptom, sign, or marker of a tumor or malignancy. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

The terms “cancer” and “malignancy” refer to an uncontrolled growth of cells which interferes with the normal functioning of the bodily organs and systems. A cancer or malignancy may be primary or metastatic, i.e. that is it has become invasive, seeding tumor growth in tissues remote from the original tumor site. A “tumor” refers to an uncontrolled growth of cells which interferes with the normal functioning of the bodily organs and systems. A subject that has a cancer is a subject having objectively measurable cancer cells present in the subject's body. Included in this definition are benign tumors and malignant cancers, as well as potentially dormant tumors and micro-metastases. Cancers that migrate from their original location and seed other vital organs can eventually lead to the death of the subject through the functional deterioration of the affected organs. Hematologic malignancies (hematopoietic cancers), such as leukemias and lymphomas, are able to e.g., out-compete the normal hematopoietic compartments in a subject, thereby leading to hematopoietic failure (in the form of anemia, thrombocytopenia, and neutropenia) ultimately causing death.

Examples of cancers include, but are not limited to, carcinomas, lymphomas, blastomas, sarcomas, and leukemias. More particular examples of such cancers include, but are not limited to, basal cell carcinoma, biliary tract cancer, bladder cancer, bone cancer, brain and CNS cancer, breast cancer, cancer of the peritoneum, cervical cancer; choriocarcinoma, colon and rectum cancer (colorectal cancer), connective tissue cancer, cancer of the digestive system, endometrial cancer, esophageal cancer, eye cancer, cancer of the head and neck, gastric cancer (including gastrointestinal cancer and stomach cancer), glioblastoma (GBM), hepatic carcinoma, hepatoma, intra-epithelial neoplasm, kidney or renal cancer, larynx cancer, leukemia, liver cancer, lung cancer (e.g., small-cell lung cancer, non-small cell lung cancer, adenocarcinoma of the lung, and squamous carcinoma of the lung), lymphoma including Hodgkin's and non-Hodgkin's lymphoma, melanoma, myeloma, neuroblastoma, oral cavity cancer (e.g., lip, tongue, mouth, and pharynx), ovarian cancer, pancreatic cancer, prostate cancer, retinoblastoma, rhabdomyosarcoma, cancer of the respiratory system, salivary gland carcinoma, sarcoma, skin cancer, squamous cell cancer, testicular cancer, thyroid cancer, uterine or endometrial cancer, cancer of the urinary system, vulval cancer; as well as other carcinomas and sarcomas, as well as B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's Macroglobulinemia), chronic lymphocytic leukemia (CLL), acute lymphoblastic leukemia (ALL), Hairy cell leukemia, chronic myeloblastic leukemia, and post-transplant lymphoproliferative disorder (PTLD), as well as abnormal vascular proliferation associated with phakomatoses, edema (such as that associated with brain tumors), and Meigs' syndrome.

In some embodiments, the carcinoma is selected from a solid tumor, including but not limited to, melanoma, prostate cancer, ovarian cancer, cervical cancer, breast cancer, lung cancer, colon cancer, kidney cancer, and head and neck cancer.

In some embodiments, the cancer or malignancy is MIC-positive (MIC+). As used herein, the terms “MIC-positive” or “sMIC+” are used to describe a cancer cell, a cluster of cancer cells, a tumor mass, or a metastatic cell that express MIC on the cell surface (membrane-bound MIC) and/or produce a sMIC protein that is released from the cancer cell(s). These terms are intended to encompass all cancer cells and/or tumor masses that shed all or part of the extracellular domain of a MIC protein into the intratumoral space or into the circulatory or lymphatic system; thus these cells may only display a MIC protein on its surface for a short time period—that is, the term encompasses cancer cells and tumor cells that shed sMIC protein or secret sMIC through exosomes or other mechanisms, regardless of whether detectable MIC protein remains present on their cell surface or not. However, any cancer cell or tumor that is capable of escaping innate immune rejection by shedding MIC is considered to be a “MIC-positive cancer” as that term is used herein. Some non-limiting examples of MIC-positive cancers include epithelial cell cancers and hematopoietic malignancies. In some embodiments, the MIC-positive cancer or malignancy can be a MIC-positive prostate cancer and/or metastasis thereof.

As used herein, a “subject” refers to a human or animal. Usually the animal is a vertebrate such as a primate, rodent, domestic animal or game animal. Primates include chimpanzees, cynomologus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments, the subject is a mammal, e.g., a primate, e.g., a human. The terms, “patient”, “individual” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used, for example, as subjects that represent animal models of, for example, various cancers. In addition, the methods described herein can be used to treat domesticated animals and/or pets. A subject can be male or female. In certain embodiments, the subject is a human.

A subject can be one who has been previously diagnosed with or identified as suffering from a sMIC+ or MIC+ cancer and in need of treatment, but need not have already undergone treatment for the sMIC+ or MIC+ cancer. Alternatively, a subject can also be one who has not been previously diagnosed as having a sMIC+ or MIC+ cancer in need of treatment. A subject can be one who exhibits one or more risk factors for a condition or one or more complications related to a sMIC+ or MIC+ cancer or a subject who does not exhibit risk factors. A “subject in need” of treatment for a sMIC+ cancer particular can be a subject having that condition or diagnosed as having that condition. In other embodiments, a subject “at risk of developing” a condition refers to a subject diagnosed as being at risk for developing the condition (e.g., a sMIC+ or MIC+ cancer).

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, reduction in free sMIC levels in the subject, alleviation of one or more symptom(s), diminishment of extent of the deficit, stabilized (i.e., not worsening) state of a cancer or malignancy, delay or slowing of tumor growth and/or metastasis, and an increased lifespan as compared to that expected in the absence of treatment. As used herein, the term “administering,” refers to providing an anti-MIC antibody described herein or a nucleic acid encoding the anti-MIC antibody as described herein into a subject. Similarly, a pharmaceutical composition comprising an anti-MIC antibody as described herein or a nucleic acid encoding the anti-MIC antibody as described herein can be administered by any appropriate route which results in an effective treatment in the subject.

The dosage ranges for an anti-MIC antibody depend upon the potency, and encompass amounts large enough to produce the desired effect e.g., reducing levels of MIC, slowing of tumor growth or a reduction in tumor size. The dosage should not be so large as to cause unacceptable adverse side effects. Generally, the dosage will vary with the age, condition, and sex of the subject and can be determined by one of skill in the art. The dosage can also be adjusted by the individual physician in the event of any complication. In some embodiments, the dosage ranges from 0.1 mg/kg body weight to 10 mg/kg body weight. In some embodiments, the dose range is from 0.5 mg/kg body weight to 5 mg/kg body weight. Alternatively, the dose range can be titrated to maintain serum levels between 1 μg/mL and 1000 μg/mL. For systemic administration, subjects can be administered a therapeutic amount, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg or more.

Administration of the doses recited above can be repeated. In fact, to the extent that clearing of free or unbound MIC can promote immune attack on a tumor, cancer cell or malignant cell, long term administration is contemplated, e.g. first to treat the tumor, cancer or malignancy itself, and then to provide continued surveillance against the development of tumor cells that gain the ability to shed MIC. In a preferred embodiment, the doses recited above are administered weekly, biweekly, every three weeks or monthly for several weeks or months. The duration of treatment depends upon the subject's clinical progress and responsiveness to treatment.

In some embodiments, a dose can be from about 0.1 mg/kg to about 100 mg/kg. In some embodiments, a dose can be from about 0.1 mg/kg to about 25 mg/kg. In some embodiments, a dose can be from about 0.1 mg/kg to about 20 mg/kg. In some embodiments, a dose can be from about 0.1 mg/kg to about 15 mg/kg. In some embodiments, a dose can be from about 1 mg/kg to about 100 mg/kg. In some embodiments, a dose can be from about 1 mg/kg to about 25 mg/kg. In some embodiments, a dose can be from about 1 mg/kg to about 20 mg/kg. In some embodiments, a dose can be from about 1 mg/kg to about 15 mg/kg. In some embodiments, a dose can be about 2 mg/kg. In some embodiments, a dose can be about 4 mg/kg. In some embodiments, a dose can be about 5 mg/kg. In some embodiments, a dose can be about 6 mg/kg. In some embodiments, a dose can be about 8 mg/kg. In some embodiments, a dose can be about 10 mg/kg. In some embodiments, a dose can be about 15 mg/kg. In some embodiments, a dose can be from about 100 mg/m² to about 700 mg/m². In some embodiments, a dose can be about 250 mg/m². In some embodiments, a dose can be about 375 mg/m². In some embodiments, a dose can be about 400 mg/m². In some embodiments, the dose can be about 500 mg/m².

In some embodiments, a dose can be administered intravenously. In some embodiments, an intravenous administration can be an infusion occurring over a period of from about 10 minute to about 4 hours. In some embodiments, an intravenous administration can be an infusion occurring over a period of from about 30 minutes to about 90 minutes.

In some embodiments, a dose can be administered weekly. In some embodiments, a dose can be administered bi-weekly. In some embodiments, a dose can be administered about every 2 weeks. In some embodiments, a dose can be administered about every 3 weeks. In some embodiments, a dose can be administered every three weeks. In some embodiments, a dose can be administered every four weeks.

In some embodiments, a total of from about 2 to about 10 doses are administered to a subject. In some embodiments, a total of 4 doses are administered. In some embodiments, a total of 5 doses are administered. In some embodiments, a total of 6 doses are administered. In some embodiments, a total of 7 doses are administered. In some embodiments, a total of 8 doses are administered. In some embodiments, a total of 9 doses are administered. In some embodiments, a total of 10 doses are administered. In some embodiments, a total of more than 10 doses are administered.

Pharmaceutical compositions containing an anti-MIC antibody can be administered in a unit dose. The term “unit dose” when used in reference to a pharmaceutical composition refers to physically discrete units suitable as unitary dosage for the subject, each unit containing a predetermined quantity of active material (e.g., an anti-MIC antibody), calculated to produce the desired therapeutic effect in association with the required physiologically acceptable diluent, i.e., carrier, or vehicle.

In some embodiments, the anti-MIC antibody provided herein or a pharmaceutical composition thereof, is administered with an immunotherapy. As used herein, “immunotherapy” refers to therapeutic strategies designed to induce or augment the subject's own immune system to fight the cancer or malignancy. Examples of an immunotherapy include, but are not limited to, adoptive cell therapies (e.g., autologous NK cells, allogeneic NK cells, autologous T cells, CAR modified T cells and CAR modified NK cells), antibodies such as check point inhibitors, and antibodies disturbing metabolic signaling, immune cytokines, such as gamma-chain family cytokines IL-2, IL15 and variants thereof, chemotoxins, radiation therapy, and epigenetic modifiers. See, e.g., Rohaan et al., Virchows Archiv 474:449-461 (2019); Magalhaes et al., Expert Opinion on Biological Therapy 19:8, 811-827 (2019) and Ott et al., 2019 ASCO Educational Book, Developmental Immunotherapy and Tumor Immunobiology, e70-78 (2020), the disclosures of which are incorporated by reference herein.

In some embodiments, the adoptive cell therapy is a T cell therapy, such as CAR T cell therapy in which T cells are removed from the subject blood, genetically modified to express a chimeric antigen receptor against a suitable target on a cancer cell) and then re-administered to the subject. See, e.g., WO2019/018603; WO2019/090003; WO2020/033927; WO2019089969; WO2015/164675; WO2016064929; WO2019/032929; WO2016/115559; WO2016/033570; WO2014/130657; WO2016028896; WO2015/090230; and WO2014/153270.

In some embodiments, the adoptive cell therapy is a cell therapy in which PBMCs are removed from the subject blood, primed to respond to a particular antigen, and then re-administered to the subject (e.g., Sipuleucel-T). (See, e.g., WO2001/039594; WO2001/074855; and WO1999/063050.)

In some embodiments, the adoptive cell therapy is an NK cell therapy. NK cells can be engineered to express a chimeric antigen receptor against a suitable target on a cancer cell and then the engineered NK cells are administered to a subject. (See, e.g., WO2006/103569; WO2018/165291; WO2016/201304; WO2017/100709; WO2019/028337; WO2016/176651 and WO2018/183385)

In some embodiments, the immunotherapy involves administration of a checkpoint inhibitor. In some embodiments, the checkpoint inhibitor is selected from inhibitors or CTLA-4, PD-1, PD-L1, PL-L2, B7-H3, B7-H4, BMA, HVEM, TIM3, GAL9, LAG3, VISTA, KIR, 2B4, CD160, CGEN-15049, CHK1, CHK2, and A2aR. In some embodiments, the immune checkpoint inhibitors include agents that inhibit CTLA-4, PD-1, PD-L1, and the like. Suitable anti-CTLA-4 therapy agents, include, for example, anti-CTLA-4 antibodies, human anti-CTLA-4 antibodies, mouse anti-CTLA-4 antibodies, mammalian anti-CTLA-4 antibodies, humanized anti-CTLA-4 antibodies, monoclonal anti-CTLA-4 antibodies, polyclonal anti-CTLA-4 antibodies, chimeric anti-CTLA-4 antibodies, ipilimumab, tremelimumab, anti-CTLA-4 adnectins, anti-CTLA-4 domain antibodies, single chain anti-CTLA-4 mAbs, heavy chain anti-CTLA-4 mAbs, light chain anti-CTLA-4 mAbs, inhibitors of CTLA-4 that agonize the co-stimulatory pathway, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP1212422B 1. Additional anti-CTLA-4 antibodies are described in U.S. Pat. Nos. 5,811,097, 5,855,887, 6,051,227, and 6,984,720; in PCT Publication Nos. WO 01/14424 and WO 00/37504; and in U.S. Publication Nos. 2002/0039581 and 2002/086014. Other anti-CTLA-4 antibodies that can be used in a method of the present invention include, for example, those disclosed in: WO 98/42752; U.S. Pat. Nos. 6,682,736 and 6,207,156; Hurwitz et al., Proc. Natl. Acad. Sci. USA, 95(17): 10067-10071 (1998); Camacho et al., J. Clin. Oncology, 22(145): Abstract No. 2505 (2004) (antibody CP-675206); Mokyr et al., Cancer Res, 58:5301-5304 (1998), U.S. Pat. Nos. 5,977,318, 6,682,736, 7,109,003, and 7,132,281.

Suitable anti-PD-1 and anti-PD-L1 therapy agents, include, for example, anti-PD-1 and anti-PD-L1 antibodies, human anti-PD-1 and anti-PD-L1 antibodies, mouse anti-PD-1 and anti-PD-L1 antibodies, mammalian anti-PD-1 and anti-PD-L1 antibodies, humanized anti-PD-1 and anti-PD-L1 antibodies, monoclonal anti-PD-1 and anti-PD-L1 antibodies, polyclonal anti-PD-1 and anti-PD-L1 antibodies, chimeric anti-PD-1 and anti-PD-L1 antibodies, anti-PD-1 adnectins and anti-PD-L1 adnectins, anti-PD-1 domain antibodies and anti-PD-L1 domain antibodies, single chain anti-PD-1 mAbs and single chain anti-PD-L1 mAbs, heavy chain anti-PD-1 mAbs and heavy chain anti-PD-L1 mAbs, and light chain anti-PD-1 mAbs and light chain anti-PD-L1 mAbs. In specific embodiments, anti-PD-1 therapy agents include nivolumab, pembrolizumab, pidilizumab, MEDI0680, and combinations thereof. In other specific embodiments, anti-PD-L1 therapy agents include atezolizumab, BMS-936559, MEDI4736, MSB0010718C, and combinations thereof.

Suitable anti-PD-1 and anti-PD-L1 antibodies are also described in Topalian, et al., Immune Checkpoint Blockade: A Common Denominator Approach to Cancer Therapy, Cancer Cell 27: 450-61 (Apr. 13, 2015), incorporated herein by reference in its entirety.

In some embodiments, the checkpoint inhibitor is Ipilimumab (Yervoy), Nivolumab (Opdivo), Pembrolizumab (Keytruda), Atezolizumab (Tecentriq), Avelumab (Bavencio), or Durvalumab (Imfinzi).

In some embodiments, provided is a method of improving treatment outcome in a subject receiving immunotherapy. The method generally includes administering an effective amount of an immunotherapy to the subject having cancer; and administering a therapeutically effective amount of a binding agent or a pharmaceutical composition thereof to the subject, wherein the binding agent specifically binds to circulating MIC; wherein the treatment outcome of the subject is improved, as compared to administration of the immunotherapy alone. In some embodiments, the binding agent comprises (i) a heavy chain variable region having the amino acid sequence set forth in SEQ ID NO: 7, and (ii) a light chain variable region having the amino acid sequence set forth in SEQ ID NO: 8, wherein the heavy and light chain framework regions are optionally modified with from 1 to 8 amino acid substitutions, deletions or insertions in the framework regions, wherein the binding agent specifically binds to MIC with a binding affinity greater than that of antibody B10G5. In some embodiments, the binding agent comprises (i) a heavy chain variable region having the amino acid sequence set forth in SEQ ID NO: 7, and (ii) a light chain variable region having the amino acid sequence set forth in SEQ ID NO: 8, wherein the binding agent specifically binds to MIC. In some embodiments, the binding agent is an antibody or an antigen-binding portion thereof. In some embodiments, the binding agent is a monoclonal antibody, a Fab, a Fab′, an F(ab′), an Fv, a disulfide linked Fc, a scFv, a single domain antibody, a diabody, a bi-specific antibody, or a multi-specific antibody.

In some embodiments, the improved treatment outcome is an objective response selected from stable disease, a partial response or a complete response as determined by standard medical criteria for the cancer being treated. In some embodiments, the improved treatment outcome is reduced tumor burden. In some embodiments, the improved treatment outcome is progression-free survival or disease-free survival.

In some embodiments, the methods further administering an anti-MIC antibody or at least four weeks, at least six weeks or at least 8 weeks after administration of chemotherapy or other treatment that impairs the immune system by depleting endogenous NK cells or T cells or their precursors. In some such embodiments, the chemotherapy or treatment is selected from the group consisting of: radiation therapy, or chemotherapy. Non-limiting examples of chemotherapeutic agents can include alkylating agents such as thiotepa and CYTOXAN™ cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK™ polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL™ paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE™ doxetaxel (Rhone-Poulenc Rorer, Antony, France); chloranbucil; GEMZAR™ gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE™ vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb™); inhibitors of PKC-alpha, Raf, H-Ras, EGFR (e.g., erlotinib (Tarceva™)) and VEGF-A that reduce cell proliferation and pharmaceutically acceptable salts, acids or derivatives of any of the above.

By “radiation therapy” is meant the use of directed gamma rays or beta rays to induce sufficient damage to a cell so as to limit its ability to function normally or to destroy the cell altogether.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### Further Numbered Embodiments

Further numbered embodiments of the disclosure are provided as follows:

Embodiment 1. An anti-MIC antibody comprising a heavy chain variable (VH) region, a light chain variable (VL) region, and an Fc domain, wherein the VH region comprises a complementarity determining region HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3, wherein the VL region comprises a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6, wherein the VH region and VL region each comprise a humanized framework region sequence, and wherein the Fc domain comprises the point mutations L234A and L235A according to the EU index.

Embodiment 2. The anti-MIC antibody of Embodiment 1, wherein the Fc domain further comprises a P329G mutation according to the EU index.

Embodiment 3. The anti-MIC antibody of Embodiment 1, wherein the Fc domain comprises the point mutations P329G, L234A, and L235A.

Embodiment 4. The anti-MIC antibody of any one of Embodiments 1-3, wherein the VH region comprises the amino acid sequence of SEQ ID NO: 7 and the VL region comprises the amino acid sequence of SEQ ID NO: 8.

Embodiment 5. The anti-MIC antibody of any one of Embodiments 1-3, wherein the humanized VH framework region is derived from a human germline gene having the amino acid sequence set forth in IMGT IGHV4-59*11, IGHV4-30-4*01 or IGHV4-30-4*08.

Embodiment 6. The anti-MIC antibody of any one of Embodiments 1-3, wherein the humanized VL framework region is derived from a human germline gene having the amino acid sequence set forth in IMGT IGKV1-5*01, IGKV1-5*02 or IGKV1-5*03.

Embodiment 7. An anti-MIC antibody comprising a heavy chain and a light chain, wherein the heavy chain comprises the amino acid sequence of SEQ ID NO: 9 comprising the point mutations L234A and L235A according to the EU index, and the light chain comprises the amino acid sequence of SEQ ID NO: 10.

Embodiment 8. An anti-MIC antibody comprising a heavy chain and a light chain, wherein the heavy chain comprises the amino acid sequence of SEQ ID NO: 9 comprising the point mutations P329G, L234A, and L235A according to the EU index, and the light chain comprises the amino acid sequence of SEQ ID NO: 10.

Embodiment 9. The anti-MIC antibody of any one of Embodiments 1-8, wherein the antibody increases Natural Killer (NK) cell activation compared to an anti-MIC antibody that does not comprise an Fc domain comprising the point mutations P329G, L234A, and/or L235A according to the EU index.

Embodiment 10. The anti-MIC antibody of any one of Embodiments 1-8, wherein the antibody increases tumor cell lysis compared to an anti-MIC antibody that does not comprise an Fc domain comprising the point mutations P329G, L234A, and/or L235A according to the EU index.

Embodiment 11. The anti-MIC antibody of any one of Embodiments 1-8, wherein the antibody abrogates MIC+ extracellular vesicles (MIC+ EV)-mediated immune suppression to a greater extent than an anti-MIC antibody that does not comprise an Fc domain comprising the point mutations P329G, L234A, and/or L235A according to the EU index.

Embodiment 12. A pharmaceutical composition comprising the anti-MIC antibody of any one of Embodiments 1-11.

Embodiment 13. A nucleic acid encoding an antibody heavy chain comprising the amino acid sequence of SEQ ID NO: 9, wherein the encoded antibody chain further comprises the point mutations (i) L234A and L235A; or (ii) P329G, L234A, and L235A.

Embodiment 14. A vector comprising the nucleic acid of Embodiment 13.

Embodiment 15. The vector of Embodiment 14, further comprising a nucleic acid sequence encoding an antibody light chain comprising the amino acid sequence of SEQ ID NO: 10.

Embodiment 16. A host cell comprising the vector of Embodiment 14 or Embodiment 15.

Embodiment 17. A method of treating a MIC+ cancer, comprising administering to a subject in need thereof a therapeutically effective amount of the anti-MIC antibody of any one of Embodiments 1 to 11 or the pharmaceutical composition of Embodiment 12.

Embodiment 18. A method of abrogating MIC-positive extracellular vesicle (MIC+ EV) induced immunosuppression in a subject suffering from a cancer, comprising administering a therapeutically effective amount of the anti-MIC antibody of any one of Embodiments 1 to 11 or the pharmaceutical composition of Embodiment 12.

Embodiment 19. The method of Embodiment 18, wherein the MIC+ EV induced immunosuppression is decreased expression of NKG2D on NK cells and/or decreased NK cell degranulation.

Embodiment 20. The method of Embodiment 17 or 18, wherein the cancer is a carcinoma or a hematologic malignancy.

Embodiment 21. The method of Embodiment 20, wherein the carcinoma is a solid tumor selected from melanoma, prostate cancer, ovarian cancer, cervical cancer, breast cancer, lung cancer, colon cancer, kidney cancer, and head and neck cancer.

Embodiment 22. The method of Embodiment 20, wherein the hematologic malignancy is a lymphoma or multiple myeloma.

Embodiment 23. The method of any one of Embodiments 17-22, further comprising administering an immunotherapy to the subject.

Embodiment 24. The method of Embodiment 23, wherein the immunotherapy comprises an adoptive cell therapy or a checkpoint inhibitor.

Embodiment 25. The method of Embodiment 24, wherein the adoptive cell therapy is selected from autologous NK cells, allogeneic NK cells, autologous T cells, CAR-modified T cells, and CAR-modified NK cells.

Embodiment 26. The method of Embodiment 24, wherein the checkpoint inhibitor is an antibody that specifically binds to human PD-1, human PD-L1, or human CTLA4.

Embodiment 27. The method of Embodiment 26, wherein the checkpoint inhibitor is pembrolizumab, nivolumab, cemiplimab, or ipilimumab.

Embodiment 28. The method of any one of Embodiments 17 to 27, wherein chemotherapy is not administered to the subject for at least four weeks prior to the administration of the anti-MIC antibody.

Embodiment 29. The method of any one of Embodiments 17 to 28, wherein the anti-MIC antibody is administered intravenously.

Embodiment 30. The method of any one of Embodiments 17 to 29, wherein the anti-MIC antibody is administered in a dose of about 0.1 mg/kg to about 10 mg/kg.

Summary of the Sequences

| Description | Sequence | SEQ ID |
|---|---|---|
| VH CDR1 | GYSITSDYA | 1 |
| VH CDR2 | GYISYSGST | 2 |
| VH CDR3 | ARGGTYFDY | 3 |
| VL CDR1 | RASAHINNW | 4 |
| VL CDR2 | DATSLES | 5 |
| VL CDR3 | QHYWSTPWT | 6 |
| VH full | EVQLQESGPG LVKPSQTLSL TCTVSGYSIT SDYAWNWIRQ | 7 |
| | PPGKGLEWIG YISYSGSTNY NPSLKSRVTI SRDTSKNQFS | |
| | LKLSSVTAAD TAVYYCARGG TYFDYWGQGT LVTVSS | |
| VL full | DVVMTQSPST LSASVGDRVT ITCRASAHIN NWLAWYQQKP | 8 |
| | GKAPKLLISD ATSLESGVPS RFSGSGSGKE YTLTISSLQP | |
| | DDFATYYCQH YWSTPWTFGQ GTKVEIK | |
| HC full | EVQLQESGPG LVKPSQTLSL TCTVSGYSIT SDYAWNWIRQ | 9 |
| | PPGKGLEWIG YISYSGSTNY NPSLKSRVTI SRDTSKNQFS | |
| | LKLSSVTAAD TAVYYCARGG TYFDYWGQGT LVTVSSASTK | |
| | GPSVFPLAPS SKSTSGGTAA LGCLVKDYFP EPVTVSWNSG | |
| | ALTSGVHTFP AVLQSSGLYS LSSVVTVPSS SLGTQTYICN | |
| | VNHKPSNTKV DKKVEPKSCD KTHTCPPCPA PELLGGPSVF | |
| | LFPPKPKDTL MISRTPEVTC VVVDVSHEDP EVKFNWYVDG | |
| | VEVHNAKTKP REEQYNSTYR VVSVLTVLHQ DWLNGKEYKC | |
| | KVSNKALPAP IEKTISKAKG QPREPQVYTL PPSRDELTKN | |
| | QVSLTCLVKG FYPSDIAVEW ESNGQPENNY KTTPPVLDSD | |
| | GSFFLYSKLT VDKSRWQQGN VFSCSVMHEA LHNHYTQKSL | |
| | SLSPG | |
| LC full | DVVMTQSPST LSASVGDRVT ITCRASAHIN NWLAWYQQKP | 10 |
| | GKAPKLLISD ATSLESGVPS RFSGSGSGKE YTLTISSLQP | |
| | DDFATYYCQH YWSTPWTFGQ GTKVEIKRTV AAPSVFIFPP | |
| | SDEQLKSGTA SVVCLLNNFY PREAKVQWKV DNALQSGNSQ | |
| | ESVTEQDSKD STYSLSSTLT LSKADYEKHK VYACEVTHQG | |
| | LSSPVTKSFN RGEC | |
| Soluble mICA | EPHSLRYNLT VLSWDGSVQS GFLAEVHLDG QPFLRCDRQK | 11 |
| | CRAKPQGQWA EDVLGNKTWD RETRDLTGNG KDLRMTLAHI | |
| | KDQKEGLHSL QEIRVCEIHE DNSTRSSQHF YYDGELFLSQ | |
| | NLETEEWTMP QSSRAQTLAM NIRNFLKEDA MKTKTHYHAM | |
| | HADCLQELRR YLKSGVVLRR TVPPMVNVTR SEASEGNITV | |
| | TCRASGFYPW NITLSWRQDG VSLSHDTQQW GDVLPDGNGT | |
| | YQTWVATRIC QGEEQRFTCY MEHSGNHSTH PVPS | |
| Soluble MICb | EPHSLRYNLM VLSQDGSVQS GFLAEGHLDG QPFLRYDRQK | 12 |
| | RRAKPQGQWA EDVLGAKTWD TETEDLTENG QDLRRTLTHI | |
| | KDQKGGLHSL QEIRVCEIHE DSSTRGSRHF YYDGELFLSQ | |
| | NLETQESTVP QSSRAQTLAM NVTNFWKEDA MKTKTHYRAM | |
| | QADCLQKLQR YLKSGVAIRR TVPPMVNVTC SEVSEGNITV | |
| | TCRASSFYPR NITLTWRQDG VSLSHNTQQW GDVLPDGNGT | |
| | YQTWVATRIR QGEEQRFTCY MEHSGNHGTH PVPS | |
| IGHV4-59*11 | QVQLQESGPG LVKPSETLSL TCTVSGGSIS SHYWSWIRQP | 13 |
| | PGKGLEWIGY IYYSGSTNYN PSLKSRVTIS VDTSKNQFSL | |
| | KLSSVTAADT AVYYCAR | |
| IGHJ4*01 | YFDYWGQGTL VTVSS | 14 |
| IGHV4-30-4*01 | QVQLQESGPG LVKPSQTLSL TCTVSGGSIS SGDYYWSWIR | 15 |
| | QPPGKGLEWI GYIYYSGSTYY NPSLKSRVTI VDTSKNQFSL | |
| | KLSSVTAADT AVYYCAR | |
| IGKV1-NL1*01 | DIQMTQSPSS LSASVGDRVT ITCRASQGIS NSLAWYQQKP | 16 |
| | GKAPKLLLYA ASRLESGVPS RFSGSGSGTD YTLTISSLQP | |
| | EDFATYYCQQ YYSTP | |
| IGKJ1*01 | WTFGQGTKVE IK | 17 |
| IGKV1-33*01 | DIQMTQSPSS LSASVGDRVT ITCQASQDIS NYLNWYQQKP | 18 |
| | GKAPKLLIYD ASNLETGVPS RFSGSGSGTD FTFTISSLQP | |
| | EDIATYYCQQ YDNLP | |
| IGKV1-5*01 | DIQMTQSPST LSASVGDRVT ITCRASQSIS SWLAWYQQKP | 19 |
| | GKAPKLLIYD ASSLESGVPS RFSGSGSGTE FTLTISSLQP | |
| | DDFATYYCQQ YNSYS | |
| VH full | GAA GTG CAG CTG CAG GAA TCT GGC CCT GGC CTC GTG | 20 |
| nucleic acid | AAG CCT TCC CAG ACC CTG TCT CTG ACC TGC ACC GTG | |
| | TCC GGC TAC TCC ATC ACC TCC GAC TAC GCC TGG AAC | |
| | TGG ATC CGG CAG CCT CCT GGC AAG GGA CTG GAA TGG | |
| | ATC GGC TAC ATC TCC TAC TCC GGC TCC ACC AAC TAC | |
| | AAC CCC AGC CTG AAG TCC AGA GTG ACC ATC TCC CGG | |
| | GAC ACC TCC AAG AAC CAG TTC TCC CTG AAG CTG TCC | |
| | TCC GTG ACC GCC GCT GAT ACC GCC GTG TAC TAC TGT | |
| | GCT AGA GGC GGC ACC TAC TTC GAC TAC TGG GGC CAG | |
| | GGC ACC CTC GTG ACC GTG TCA TCT | |
| VL full | GAC GTC GTG ATG ACC CAG TCC CCC TCC ACA CTG TCT | 21 |
| nucleic acid | GCC TCT GTG GGC GAC AGA GTG ACC ATC ACC TGT CGG | |
| | GCC TCC GCC CAC ATC AAC AAC TGG CTG GCC TGG TAT | |
| | CAG CAG AAG CCC GGC AAG GCC CCT AAG CTG CTG ATC | |
| | TCT GAT GCC ACC TCC CTG GAA TCC GGC GTG CCC TCC | |
| | AGA TTC TCC GGC TCT GGC TCT GGC AAG GAG TAT ACC | |
| | CTG ACC ATC AGC TCC CTG CAG CCC GAT GAC TTC GCC | |
| | ACC TAC TAC TGC CAG CAC TAC TGG TCC ACC CCC TGG | |
| | ACC TTT GGC CAA GGC ACC AAG GTG GAA ATC AAG | |

### EXAMPLES

### Example 1: Materials and Methods

CHO cell culture: Chinese hamster ovary cells (CHO) were cultured in Ham's F12 medium supplemented with 10% FCS, L-glutamine and pyruvate. A MICA*008 expression plasmid was constructed by subcloning cDNA of full length MICA*008 into pcDNA3. The MICA expression plasmid was mixed (9:1 ratio) with a vector conferring resistance to puromycin. CHO cells were transfected with this mixture using Lipofectamine 2000. Stable transfectants were generated by culture of transfected CHO cells in selective medium (8 ug/mL puromycin; Calbiochem).

Exosome preparation: CHO cells were plated in 12×150 mm plates and allowed to rest for 2 days. Cells were then washed with PBS and EV-free media (DMEM/F12 supplemented with 1% FEBS) was added to each plate. Cultures were incubated for 3 days to accumulated vesicles in the supernatant. Supernatants were collected and centrifuged sequentially as follows:
(a) 200×g (1,500 rpm benchtop) at 4° C. for 5 min Supernatant was collected and pellet was discarded;
(b) Repeat 200×g (1,500 rpm) at 4° C. for 5 min. Supernatant was collected and pellet was discarded;
(c) 500×g (2,000 rpm benchtop) at 4° C. for 10 min. Supernatant was collected and pellet was discarded
(d) Repeat 500×g (2,000 rpm) at 4° C. for 10 min. Supernatant was collected and pellet was discarded
(e) 10,000×g (9,100 rpm Sorvall) at 4° C. for 30 min.

Supernatants were then ultracentrifuged at 100,000×g (24,000 rpm, ultracentrifuge) for 2 h at 4° C. with no brake. Pellets were carefully solubilized in the appropriate volume, for example, in 500 μL of HBS. Add buffer in several steps to recover the pellet by adding a small amount of buffer and incubating for 20 min on ice so that the pellet dissolved progressively. EVs were then resuspended and additional HBS was added to bring to the final volume. Exosomes are stored in aliquots at −80° C. in a box and −20° C. for a few days. Exosomes were then isolated from parental or transfected CHO cell supernatants. Adherent monolayers were washed extensively with PBS and re-cultured in fresh medium (no serum) for 24 hours. Cell culture media were collected and centrifuged twice for 10 min at 300×g to remove cell debris and then centrifuged for 30 min at 10,000×g and for 2 hr at 100,000×g sequentially.

NK cell isolation: Thawed PBMC from healthy donors were cultured in RPMI 10% FCS, cells left in culture overnight. The cells were then plated with irradiated 221 or 8866 feeder cells in RPMI/FCS/HS. Cells were stimulated with 50 IU/ml of IL-2.

Detection of cell surface markers: IL-2-activated Human NK cells were stimulated with IL-2 for 3 days (50 units/mL; R&D Systems) and were then incubated in 96 well flat-bottomed plates for 24 hours with varying amounts of CHO-derived exosomes. Cells were assayed for MIC and/or NKG2D expression by flow cytometry using a FACScan cytometer.

Cytotoxicity: K562 target cells were washed, counted, and resuspended at 100,000 cells/mL in RPMI with 10% FCS. 1 μg/mL calcein was used to stain the K562 cells for 30 minutes (37° C.) with the cap open. The tube was inverted every 15 minutes to make sure that the cells were stained homogenously. Cells were centrifuged at 1000 rpm for 5 minutes at room temperature, discarding the supernatant. Cells were washed twice at 1000 rpm for 5 mins at room temp, discarding the supernatant. RPM with 10% FCS was added to achieve 10,000 cells/100 μL concentration and cells were left in the tube for 1 h 30 min at 37° C. with the cap open (protect from light exposure). For cytotoxicity assays, 10,000 K562 cells were added to NK cells in a U-bottomed 96 well plate.

### Example 2: Characterization of Binding Affinity of SYB-011

Anti-MIC antibodies were tested for activity in the presence of MICA*008-expressing EVs. Previously published data with an anti-MIC antibody (6E1) indicate the therapeutic effect of the anti-MIC antibody is dependent on Fc receptor cross-linking in order to trigger NK cell activation (Ref). This study demonstrated that soluble MICA (sMICA) formed complexes with the 6E1 antibody and that these sMICA-6E1 immune complexes reversed the immunosuppressive activities of sMICA by activating NKG2D through a Fc receptor-dependent mechanism. sMICA-6E1 complexes (wherein the 6E1 antibody comprised wild-type Fc effector function) induced IFN-γ and TNF-α secretion by NK cells in the absence of tumor cells. In contrast, immune complexes formed with an effectorless Fc mutant of 6E1 (N297G) did not.

To investigate the requirement for Fc cross-linking for immune activation, a variant of the previously described SYB-010 anti-MIC antibody was produced. This variant comprises the same antigen-binding domain sequences as SYB-010, but further comprises an Fc domain with 3 point mutations that eliminate Fc receptor binding and abrogate Fc cross-linking—P329G, L234A, and L235A. This triple mutation in the Fc domain is referred to as the PG-LALA mutation and has been previously described to reduce Fc binding to complement components, to reduce binding to FcγRs, and to reduce complement-dependent cytotoxicity (See Lo et al., J Bio Chem, 292:9(2017); 3900-3908). The anti-MIC antibody comprising the PG-LALA mutation is referred to herein as SYB-011.

Experiments were performed to characterize the MIC and Fc receptor binding properties of SYB-011 in comparison to SYB-010. Table 1 provides the binding affinities for each antibody to MICB*005 and MICA*008.

TABLE 1
Binding Affinities of SYB-010 and SYB-011 to MICA*008 and MICB*005

| Antibody | Ligand | ka (1/Ms) | kd (1/s) | KD (M) | Rmax (RU) | Chi² (RU²) |
|---|---|---|---|---|---|---|
| SYB-010 | MICB*005 | 1.88E+06 | 3.94E−02 | 2.10E−08 | 53.94 | 0.809 |
| SYB-010 | MICA*008 | 6.39E+05 | 6.27E−04 | 0.98E−09 | 72.87 | 0.0825 |
| SYB-011 | MICB*005 | 4.74E+06 | 9.39E−02 | 1.98E−08 | 97.63 | 2.02 |
| SYB-011 | MICA*008 | 5.96E+05 | 6.17E−04 | 1.04E−09 | 131.1 | 0.128 |

As shown, SYB-010 and SYB-011 bind to MICA*008 and MICB*005 with very similar affinities, demonstrating that the SYB-011 Fc mutant retains similar ligand binding properties as the SYB-010 Fc wildtype.

Table 2 provides the binding affinities for each receptor to Fcγ receptors.

TABLE 2
Binding Affinities of SYB-010 and SYB-011 to Fcγ Receptors

| Antibody | FcR | ka (1/Ms) | kd (1/s) | KD (M) | Rmax (RU) | Chi² (RU²) |
|---|---|---|---|---|---|---|
| SYB-010 | Human CD64 | 8.25E+05 | 1.33E−03 | 1.61E−09 | 90.1 | 1.27E+00 |
| | (FcγRI) | | | | | |
| SYB-010 | Human CD16a | 3.60E+05 | 3.28E−02 | 9.11E−08 | 66.4 | 6.80E−01 |
| | (FcγRIIIA) | | | | | |
| SYB-010 | Human CD32a | 5.00E+05 | 7.72E−01 | 1.54E−06 | 72.9 | 2.23E−02 |
| | (FcγRIIA) | | | | | |
| SYB-011 | Human CD64 | ND | ND | ND | ND | NA |
| | (FcγRI) | | | | | |
| SYB-011 | Human CD16a | ND | ND | ND | ND | NA |
| | (FcγRIIIA) | | | | | |
| SYB-011 | Human CD32a | ND | ND | ND | ND | NA |
| | (FcγRIIA) | | | | | |

ND = not detectable;
NA = not applicable

SYB-010 binds to all Fc receptors tested. SYB-011 does not bind any of the Fc receptors, as expected based on the introduction of the PG-LALA mutation.

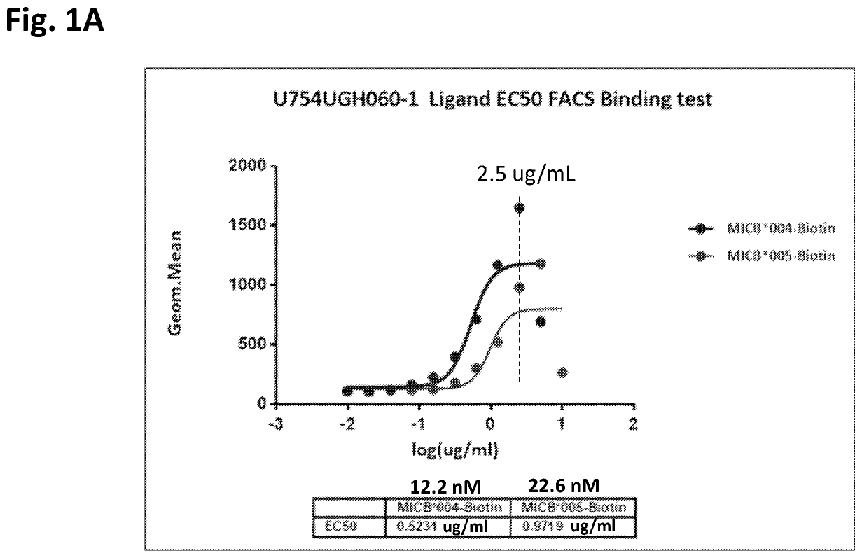

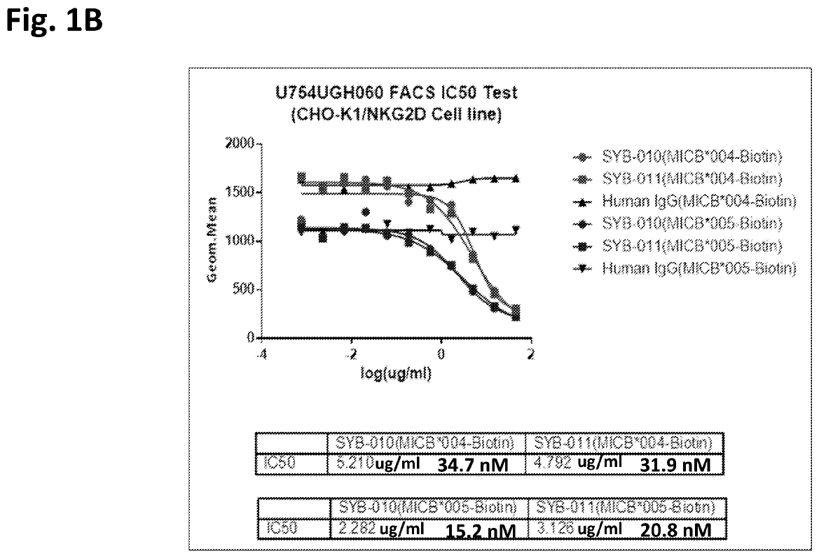

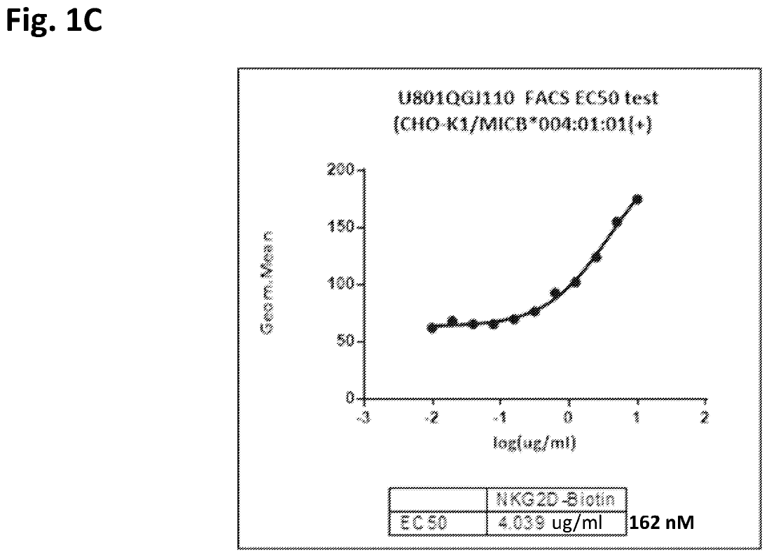

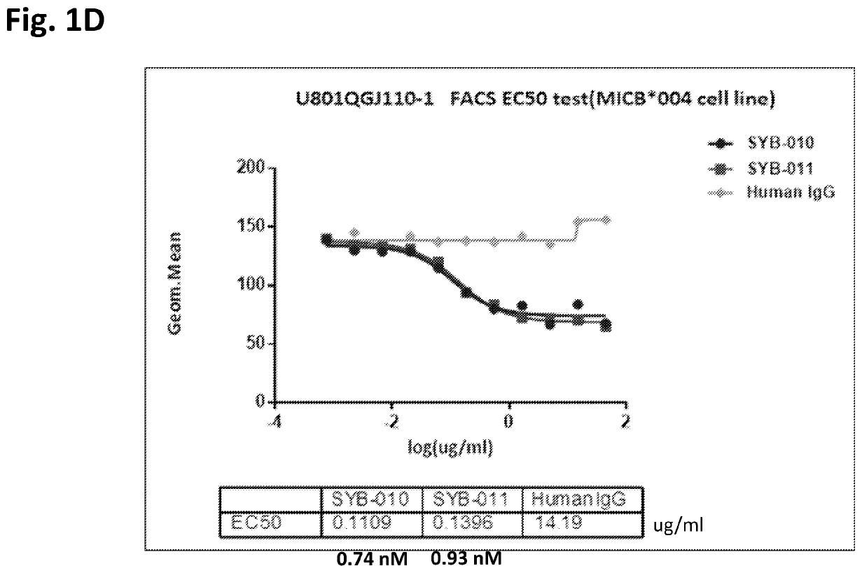

Additional experiments were performed to assess the effects of SYB-010 and SYB-011 on the binding between MICB and NKG2D. The results of this experiment are shown in FIG. 1A-FIG. 1D. As shown in FIG. 1A, biotinylated MICB bound to NKG2D expressed on the membrane of CHO cells. As shown in FIG. 1B, SYB-010 and SYB-011 blocked the binding between biotinylated MICB and NKG2D expressed on the membrane of CHO cells. As shown in FIG. 1C, biotinylated extracellular domain of NKG2D bound to MICB expressed on the membrane of CHO cells. As shown in FIG. 1D, biotinylated extracellular domain of NKG2D bound to MICB expressed on the membrane of CHO cells. SYB-010 and SYB-011 blocked the binding between biotinylated extracellular domain of NKG2D and MICB expressed on the membrane of CHO cells.

### Example 3: SYB-011 Reverses MIC+ EV-Induced NKG2D Downregulation in NK Cells

Experiments were performed to determine whether Fc receptor crosslinking was required for the function of the SYB-010 antibody. If Fc receptor cross-linking was not required, it was expected that the effectorless mutant of SYB-010, SYB-011, would have similar biological activity to SYB-010. If cross-linking was required, as suggested by previous studies, it was expected that SYB-011 would demonstrate decreased biological activity compared to SYB-010. Surprisingly, and in contrast to what is suggested by previously published data, SYB-011 demonstrated superior biological activity compared to SYB-010.

Briefly, exosomes (EVs) were isolated from wildtype parental CHO cells (CHOP) or from CHO cells transfected with a MICA*008 expression vector (*008). 3 μL of these EVs were then incubated with 5 μg/mL of one of three antibodies for approximately 20 minutes:
(a) SYB-010—Human effector competent IgG1 targeting MICA and MICB.
(b) SYB-011—Human effector incompetent IgG1 version of SYB-010; or
(c) 3F9—Anti-shedding antibody to MICA/B; effector competent murine IgG2a.

The resulting EV/antibody complexes were then cultured with IL-2 stimulated donor NK cells overnight. The next day, calcein-loaded K562 tumor target cells were added to culture at a ratio of 3:1 effector:target cell and the combination was cultured for 2 hours for NKG2D or CD107a staining or 4 hours for cytotoxicity readouts. NK cell phenotype and function were assessed by FACS for NKG2D expression, MIC expression, CD107a expression (as a measure of NK cell degranulation). K562 cell cytotoxicity was measured by calcein release.

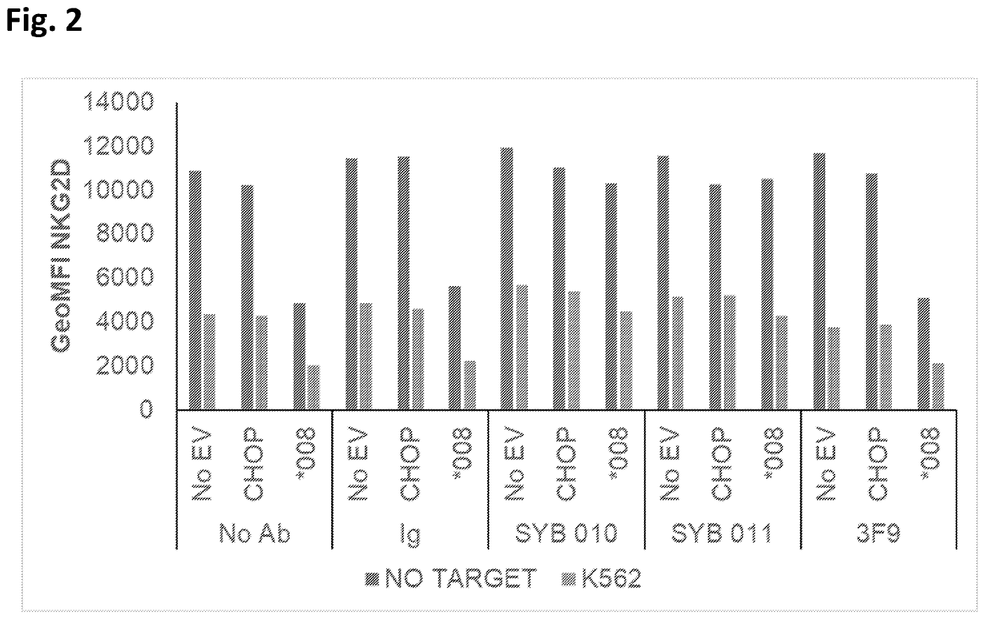

FIG. 2 shows the effect of these conditions on the expression of NKG2D on the donor NK cells. As shown with the control conditions of no antibody or control IgG incubation with the EVs, the presence of MIC+ EVs reduced NKG2D expression. This effect was seen with or without the presence of tumor cells, indicating that the MIC+ EVs are sufficient to drive the decreased NKG2D expression. Both SYB-010 and SYB-011 prevented MIC+ EV-induced NKG2D downregulation. As expected, the 3F9 antibody (which blocks MIC shedding and is not expected to play a role in exosomally released MIC) did not reverse NKG2D downregulation.

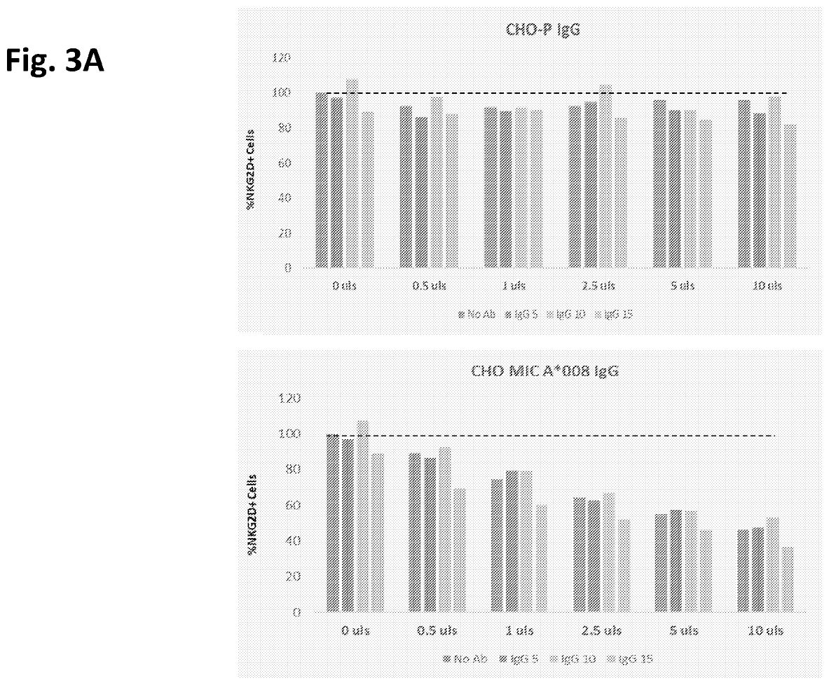

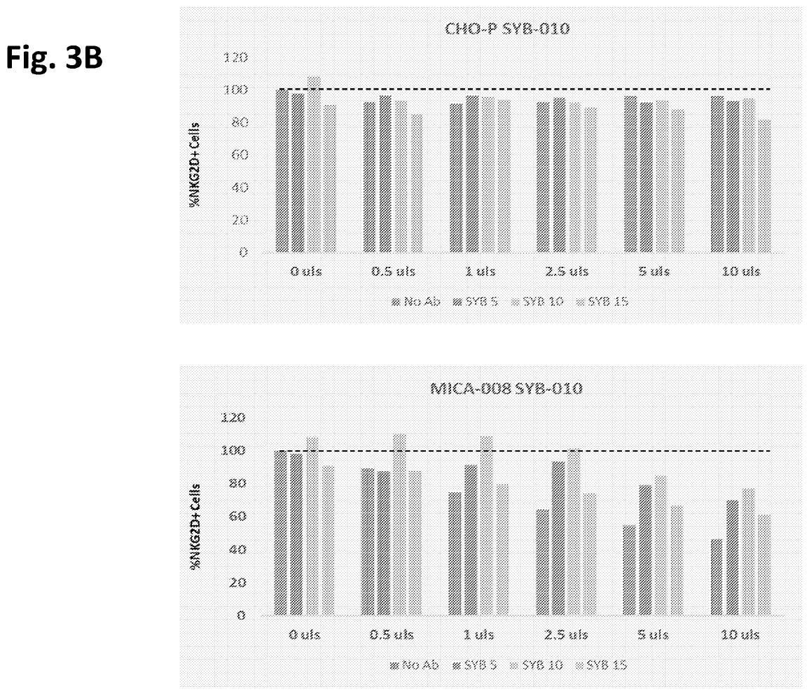

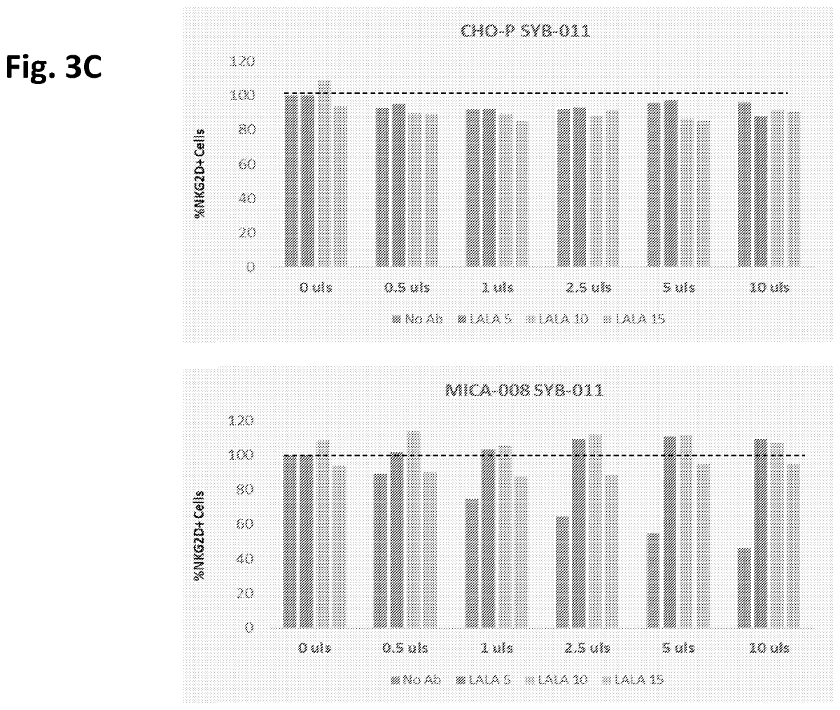

Additional experiments were performed to assess the effects of increasing amounts of MIC+ EVs on NKG2D expression and the ability of SYB-010 and SYB-011 to reverse resulting downregulation of NKG2D. The results of this experiment are shown in FIG. 3A-FIG. 3C. As shown in FIG. 3A, complexes of CHO parental EVs and control IgG had no effect on NKG2D expression, while complexes of MIC+ EVs and control IgG reduced NKG2D expression in a dose dependent manner. As shown in FIG. 3B, SYB-010 reversed the MIC+ EV-induced NKG2D downregulation but only when higher amounts of the antibody were used to generate the antibody-EV complexes. Furthermore, NKG2D expression was only partially restored when greater amounts of the antibody-EV complexes were added to the co-culture. However, as shown in FIG. 3C, SYB-011 was able to reverse the MIC+ EV-induced NKG2D downregulation even at the lowest dose of antibody used. Furthermore, NKG2D expression was restored to control levels independent of the amount of complexes added to the co-cultures.

### Example 4: SYB-011 Prevents MIC+-EV Induced Upregulation of MIC on NK Cells

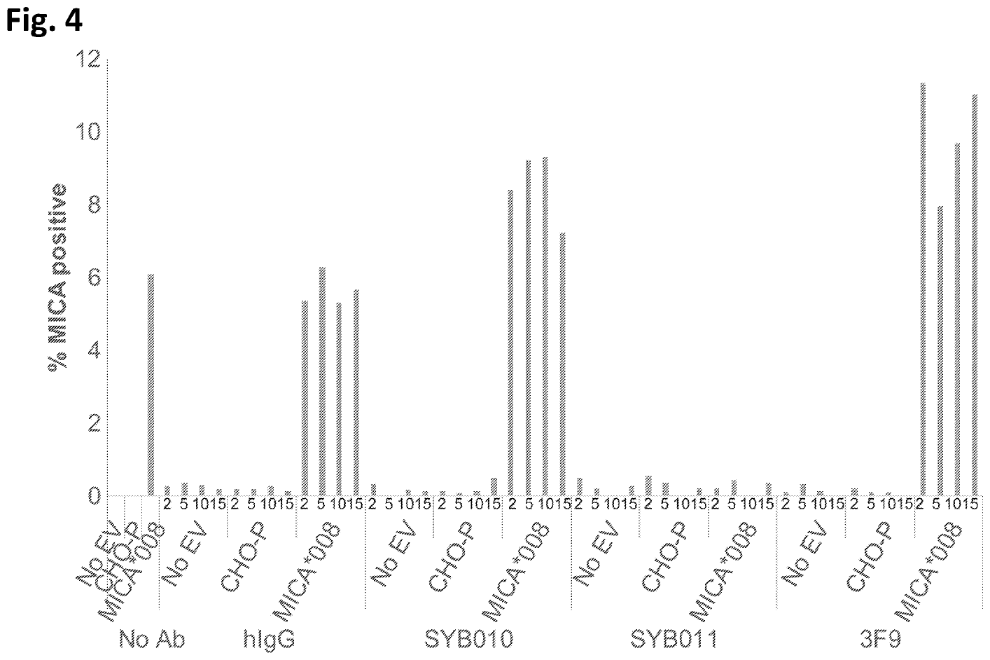

Experiments were performed as described in Example 3 and the surface expression of MIC on NK cells was determined by FACS. As shown in FIG. 4, incubation of NK cells with MIC+ EVs, but not control EVs, resulted in an increase in the percentage of MIC+ NK cells (blue bars). Pre-incubation with SYB-010 or 3F9 with MIC+ EVs further increased the increase in MIC+ NK cells. However, pre-incubation with SYB-011 prevented MIC+-EV associated increase in MIC expression on NK cells.

### Example 5: SYB-011 Prevents MIC+-EV Induced Inhibition of NK Cell Degranulation and Target Cell Cytotoxicity

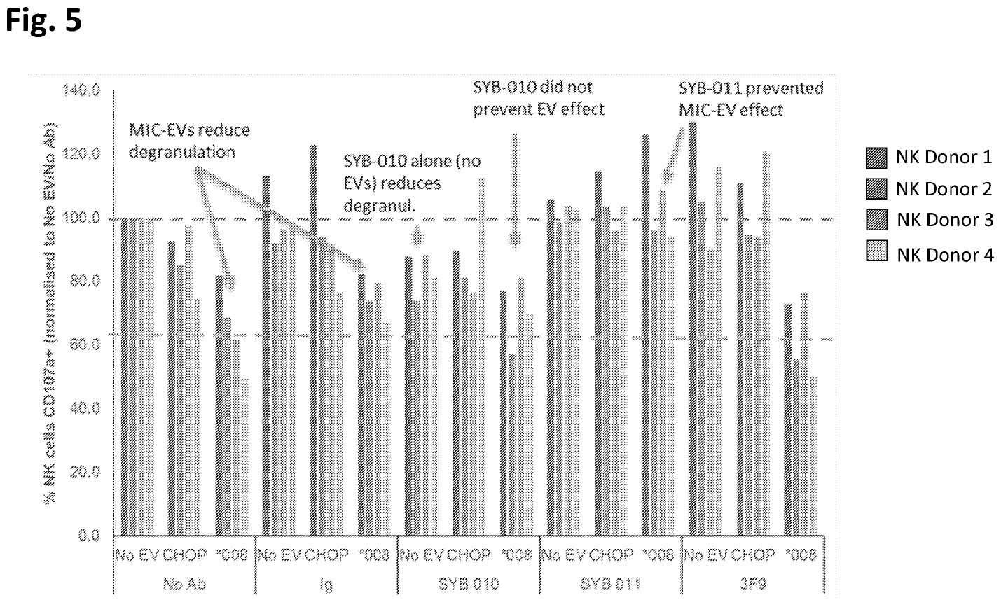

Experiments were performed as described in Example 3 with 3 μL EVs and 5 μg/mL of antibodies. NK cell degranulation was measured by CD107a expression by FACS. The results with four different NK cell donors are provided in FIG. 5. As shown, MIC+ EVs reduced NK degranulation in all donors. Further, SYB-010 was not able to reverse the MIC+-EV induced reduction in NK cell degranulation. However, SYB-011 was able to prevent the MIC+-EV induced reduction in degranulation in all donors.

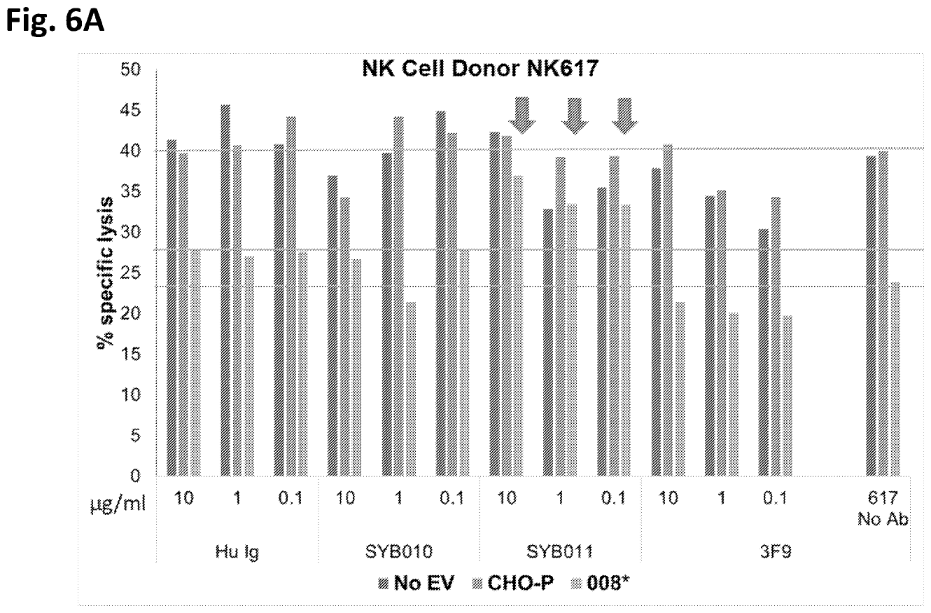

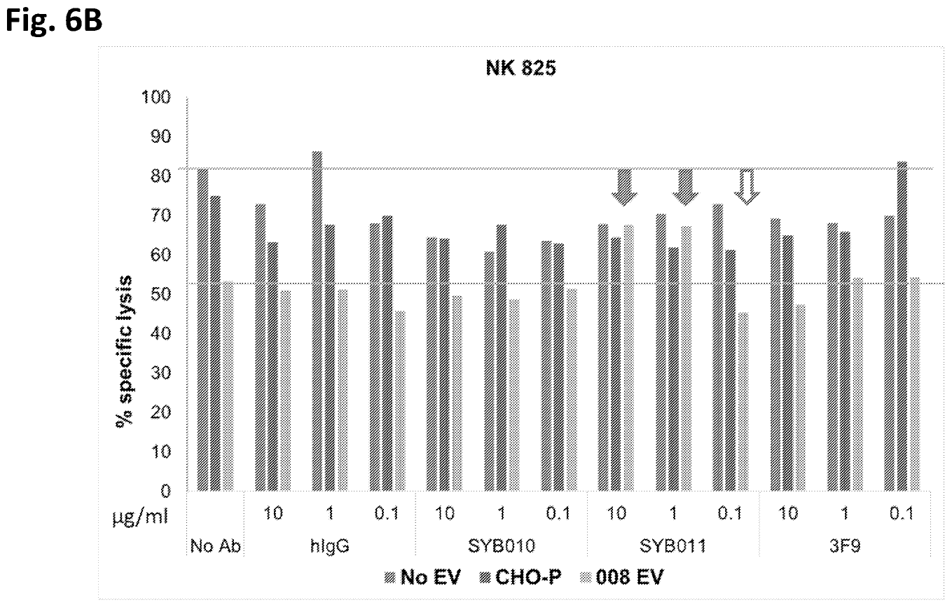

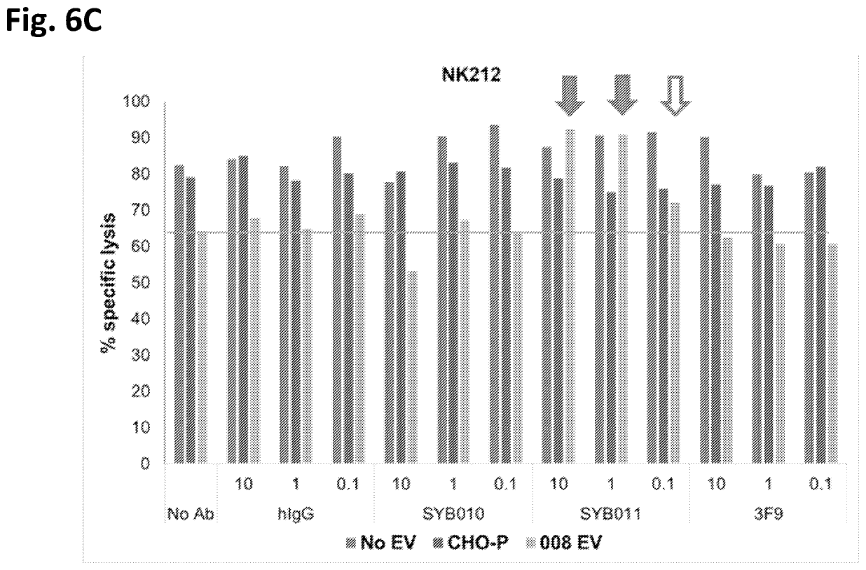

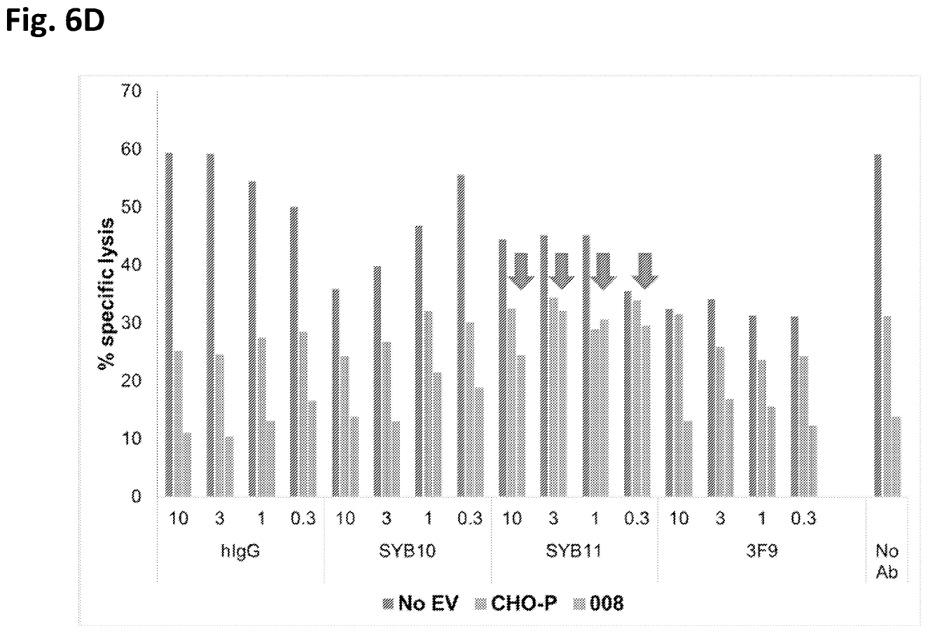

These effects on NK cell degranulation correlated with the effects on target cell cytolysis. As shown in FIG. 6A-FIG. 6D, MIC+ EVs reduce the ability of NK cells to kill K562 target cells (grey bars). Further, SYB-011, but not SYB-010 or 3F9, prevent inhibition of NK cell cytotoxicity. Results are shown for four different NK cell donors—FIG. 6A=Donor 617 (2 μL EVs, E:T ratio of 3:1); FIG. 6B=Donor 825 (3 μL EVs, E:T ratio of 3:1); FIG. 6C=Donor 212 (3 μL EVs, E:T ratio of 5:1); FIG. 6D=Donor 735 (2 μL EVs, E:T ratio of 3:1).

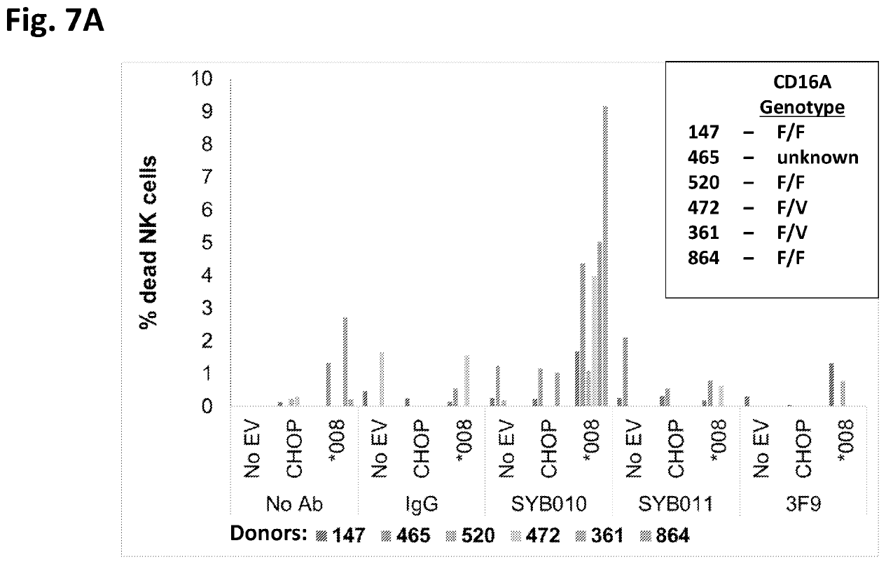

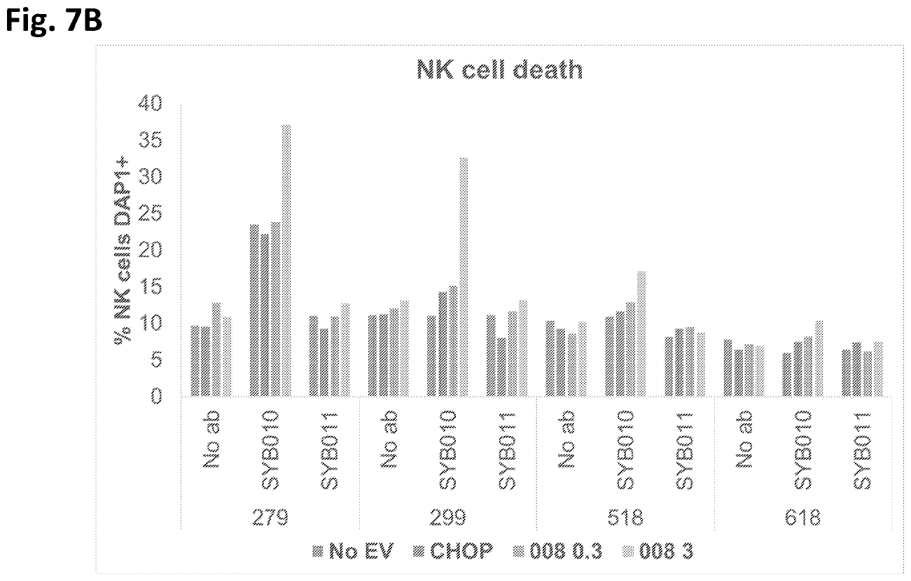

Analysis was also performed to assess the effects of SYB-010 and SYB-011 on NK cell death. The results of this analysis are shown in FIG. 7A-FIG. 7B. As shown in FIG. 7A-FIG. 7B, SYB-010, not SYB-011, was associated with increased NK cell death across donors in the presence of MIC+ EVs. Donor 279 in FIG. 7B had increased SYB-010-associated NK cell death regardless of the presence of MIC+ EVs.

### Example 6: SYB-011 Reverses MIC+-EV Mediated Suppression of NK Cell Degranulation

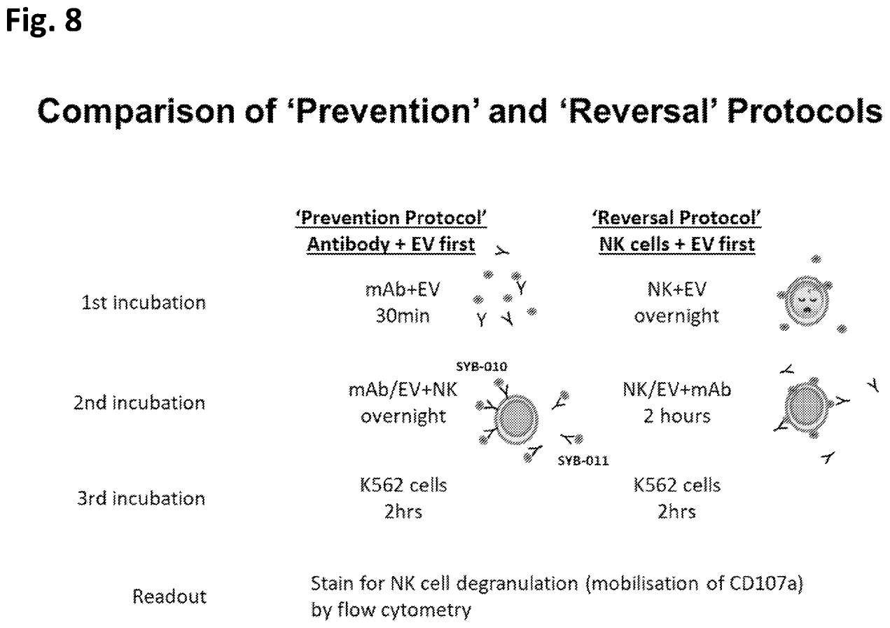

A comparison of protocols to assess “prevention” of NK cell degranulation and “reversal” of NK cell degranulation is provided in FIG. 8. Briefly, to assess the ability of antibodies to reverse MIC+-EV mediated suppression of NK cell degranulation, the monoclonal antibodies are added after the NK cells have been incubated with MIC+ EVs overnight rather than precomplexing the antibodies and EVs prior to co-culture with NK cells.

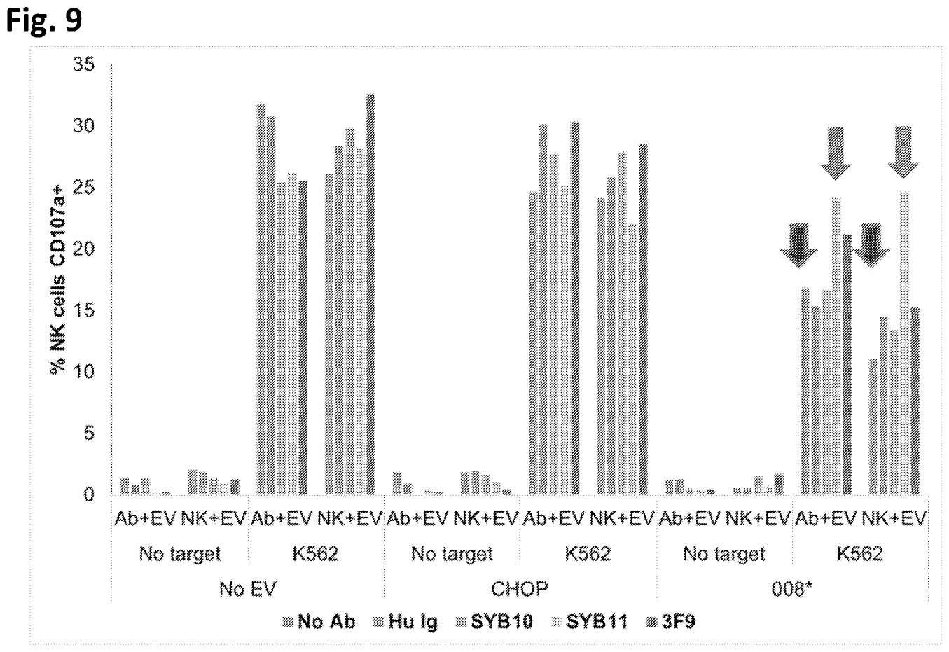

As shown in FIG. 9, NK cell degranulation induced by target cells is inhibited by MIC+ EVs (red arrows). Furthermore, SYB-011 was able to reverse the inhibition of NK cell degranulation (green arrows), while SYB-010 was not able to do so.

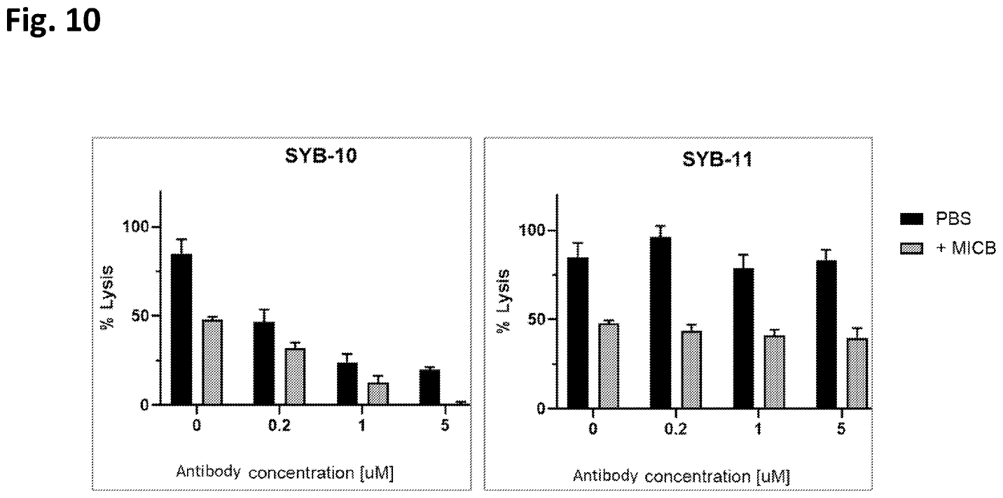

Analysis was also performed to assess the effects of SYB-010 and SYB-011 on NK cell lysis of target cells. As shown in FIG. 10, SYB-010 caused a dose-dependent reduction in cytotoxicity of NK cells. Such effect was more significant in NK cells pre-treated with MIC+ EVs.

### Example 7: SYB-011 Blocks NKG2D Downregulation after NK Cell Incubation with Target Cells

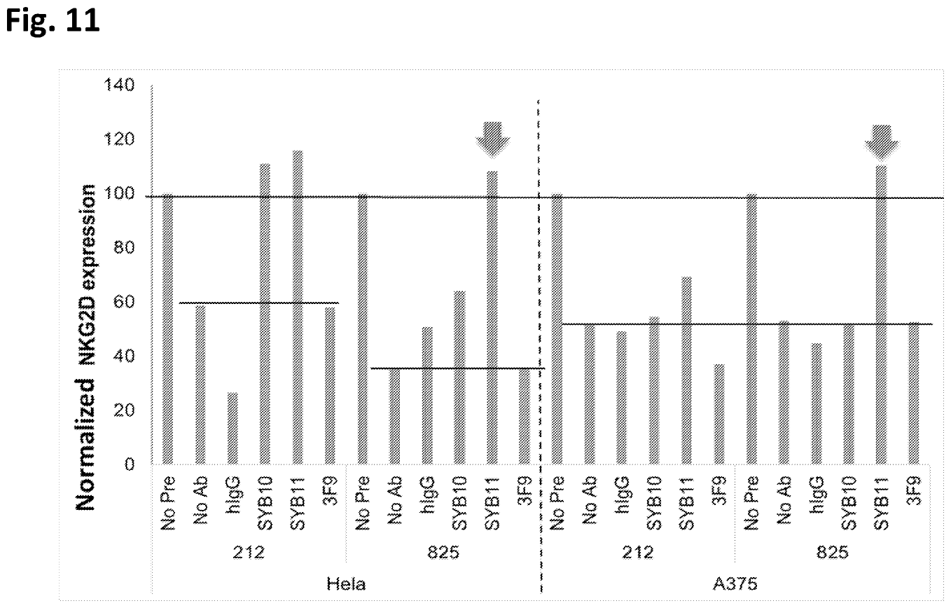

Experiments were performed to assess the effects of anti-MIC antibodies on NKG2D expression on NK cells incubated with tumor cells expressing endogenous MIC rather than with exogenously added MIC+ exosomes and tumor cells. FIG. 11 demonstrates that NK cells from two donors pre-incubated overnight in the presence of either HeLa cells or A375 cells (No Ab) reduces the expression of NKG2D compared with NK cells alone (No Pre). In the presence of both tumor cell lines, SYB-011 but not SYB-010 prevented the decrease of NKG2D on NK cells from donor 825. Both SYB-011 and SYB-010 prevented HeLa cell-induced loss of NKG2D on NK cells from donor 212. Only SYB-011 partially prevented A375 cell-mediated loss of NKG2D on NK cells from donor 212. A375 cells do not have MICA5.1 family alleles, but rather have alleles for MICA9 and MICA6 families. MICA9 family includes MICA*002 (to which SYB-011 does not bind), and MICA6 family members include MICA*004 and *009 (to which SYB-011 does bind). These results therefore suggest that the effects of SYB-011 are not limited to MICA5.1 family members.

### Example 8: SYB-011 Reverses MIC+ EV-Induced NKG2D Downregulation in T Cells and Prevents MIC+-EV Induced Upregulation of MIC on T Cells

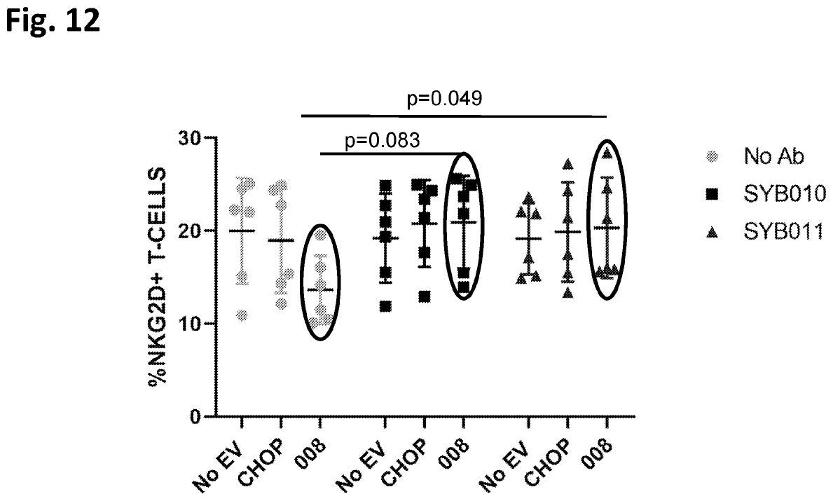

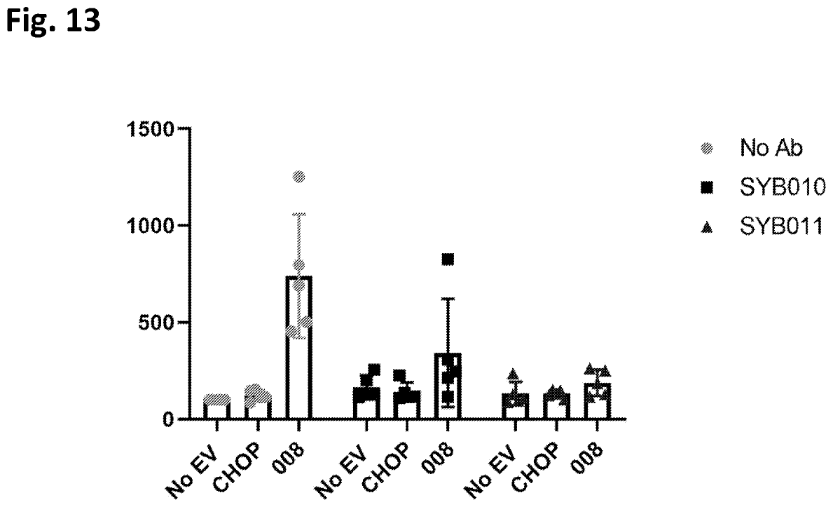

Experiments were performed as described in Example 3 but in T cells instead of NK cells. The expression of NKG2D and MIC was determined by FACS. As shown in FIG. 12, both SYB-010 and SYB-011 prevented MIC+ EV-induced NKG2D downregulation in T cells. As shown in FIG. 13, incubation of T cells with MIC+ EVs, but not control EVs, resulted in an increase in the percentage of MIC+ T cells. Pre-incubation with SYB-011, not SYB-010, prevented such MIC+ EV-associated increase in MIC expression on T cells.

### Example 9: SYB-011 Inhibits Tumor Growth In Vivo

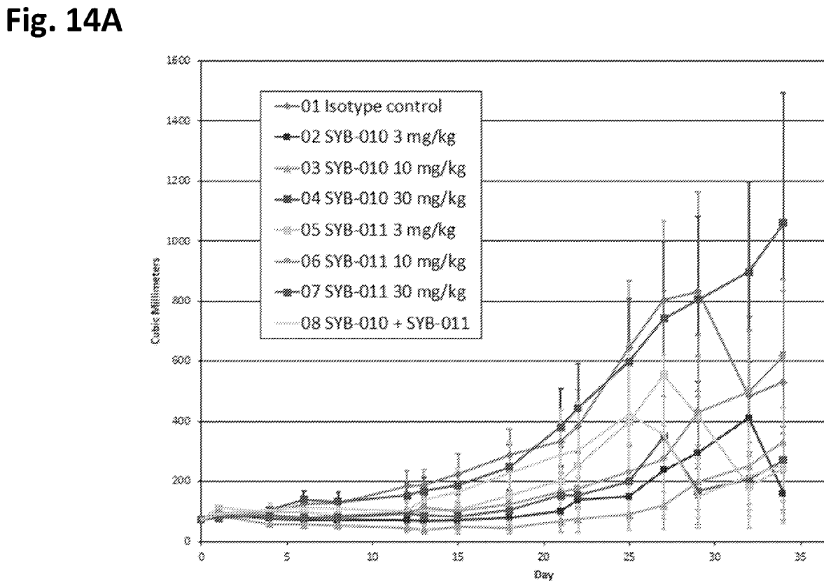

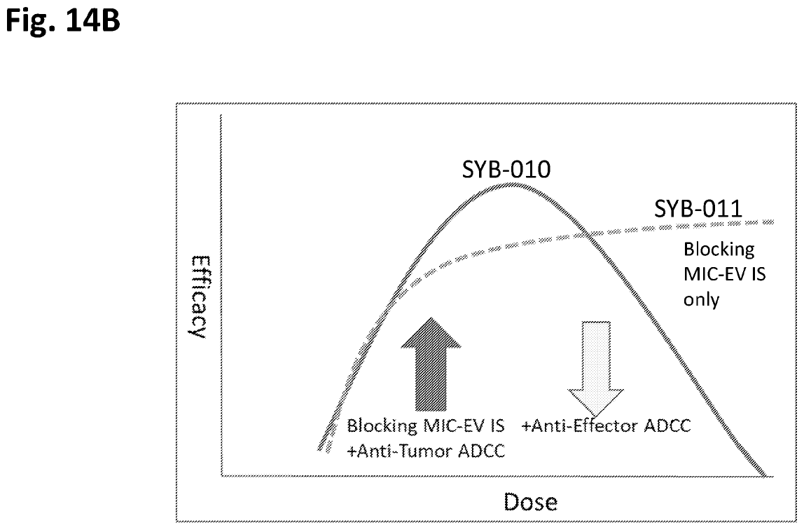

Experiments were performed to assess the effects of anti-MIC antibodies in vivo. SYB-010 and SYB-011 were administered in a MC38-MICA*008 syngeneic model twice per week for four weeks at different doses. As shown in FIG. 14A, SYB-011 inhibited tumor growth at all three doses when compared to the control group. In contrast, SYB-010 only showed strong efficacy at lower doses. The bell-shaped dose-response observed for SYB-010, which is further illustrated in FIG. 14B, suggested that SYB-010 might cause anti-effector cell ADCC at higher doses.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

1. An anti-MIC antibody comprising a heavy chain variable (VH) region, a light chain variable (VL) region, and an Fc domain,
wherein the VH region comprises a complementarity determining region HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3,
wherein the VL region comprises a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6,
wherein the VH region and VL region each comprise a humanized framework region sequence, and
wherein the Fc domain comprises the point mutations P329G, L234A, and L235A according to the EU index.

2. The anti-MIC antibody of claim 1, wherein the VH region comprises the amino acid sequence of SEQ ID NO: 7 and the VL region comprises the amino acid sequence of SEQ ID NO: 8.

3. The anti-MIC antibody of claim 1, wherein the humanized VH framework region is derived from a human germline gene having the amino acid sequence set forth in IMGT IGHV4-59*11, IGHV4-30-4*01 or IGHV4-30-4*08.

4. The anti-MIC antibody of claim 1, wherein the humanized VL framework region is derived from a human germline gene having the amino acid sequence set forth in IMGT IGKV1-5*01, IGKV1-5*02 or IGKV1-5*03.

5. An anti-MIC antibody comprising a heavy chain and a light chain, wherein the heavy chain comprises the amino acid sequence of SEQ ID NO: 9 comprising the point mutations P329G, L234A, and L235A according to the EU index, and the light chain comprises the amino acid sequence of SEQ ID NO: 10.

6. The anti-MIC antibody of claim 1, wherein the antibody increases Natural Killer (NK) cell activation compared to an anti-MIC antibody that does not comprise an Fc domain comprising the point mutations P329G, L234A, and/or L235A according to the EU index.

7. The anti-MIC antibody of claim 1, wherein the antibody increases tumor cell lysis compared to an anti-MIC antibody that does not comprise an Fc domain comprising the point mutations P329G, L234A, and/or L235A according to the EU index.

8. The anti-MIC antibody of claim 1, wherein the antibody abrogates MIC+ extracellular vesicles (MIC+ EV)-mediated immune suppression to a greater extent than an anti-MIC antibody that does not comprise an Fc domain comprising the point mutations P329G, L234A, and/or L235A according to the EU index.

9. A pharmaceutical composition comprising the anti-MIC antibody of claim 1.

10. A nucleic acid encoding an antibody heavy chain comprising the amino acid sequence of SEQ ID NO: 9, wherein the encoded antibody chain further comprises the point mutations P329G, L234A, and L235A.

11. A vector comprising the nucleic acid of claim 10.

12. The vector of claim 11, further comprising a nucleic acid sequence encoding an antibody light chain comprising the amino acid sequence of SEQ ID NO: 10.

13. A host cell comprising the vector of claim 11.

14. A method of treating a MIC+ cancer, comprising administering to a subject in need thereof a therapeutically effective amount of the anti-MIC antibody of claim 1.

15. A method of abrogating MIC-positive extracellular vesicle (MIC+ EV) induced immunosuppression in a subject suffering from a cancer, comprising administering a therapeutically effective amount of the anti-MIC antibody of any one of claim 1.

16. The method of claim 15, wherein the MIC+ EV induced immunosuppression is decreased expression of NKG2D on NK cells and/or decreased NK cell degranulation.

17. The method of claim 14, wherein the cancer is a carcinoma or a hematologic malignancy.

18. The method of claim 17, wherein the carcinoma is a solid tumor selected from melanoma, prostate cancer, ovarian cancer, cervical cancer, breast cancer, lung cancer, colon cancer, kidney cancer, and head and neck cancer.

19. The method of claim 17, wherein the hematologic malignancy is a lymphoma or multiple myeloma.

20. The method of claim 14, further comprising administering an immunotherapy to the subject.

21. The method of claim 20, wherein the immunotherapy comprises an adoptive cell therapy or a checkpoint inhibitor.

22. The method of claim 21, wherein the adoptive cell therapy is selected from autologous NK cells, allogeneic NK cells, autologous T cells, CAR-modified T cells, and CAR-modified NK cells.

23. The method of claim 21, wherein the checkpoint inhibitor is an antibody that specifically binds to human PD-1, human PD-L1, or human CTLA4.

24. The method of claim 23, wherein the checkpoint inhibitor is pembrolizumab, nivolumab, cemiplimab, or ipilimumab.

25. The method of claim 14, wherein chemotherapy is not administered to the subject for at least four weeks prior to the administration of the anti-MIC antibody.

26. The method of claim 14, wherein the anti-MIC antibody is administered intravenously.

27. The method of claim 14, wherein the anti-MIC antibody is administered in a dose of about 0.1 mg/kg to about 10 mg/kg.
